(19) United States
(12) Patent Application Publication
(10) Pub. No.: US 2020/0181101 A1
(43) Pub. Date: Jun. 11, 2020

(54) BENZAMIDE INHIBITORS OF BACTERIAL LIPOPROTEIN SIGNAL PEPTIDASE

(71) Applicant: The Scripps Research Institute, La Jolla, CA (US)

(72) Inventors: Dennis W. Wolan, La Jolla, CA (US); Seiya Katamura, San Diego, CA (US)

(21) Appl. No.: 16/628,484

(22) PCT Filed: Jul. 3, 2018
(86) PCT No.: PCT/US18/40693

§ 371 Date: Jan. 3, 2020

Related U.S. Application Data
(60) Provisional application No. 62/528,759, filed on Jul. 5, 2017.

(51) Int. Cl. C07D 285/135; G01N 33/50; G01N 33/542; A61P 31/04

(52) U.S. Cl. CPC C07D 285/135; G01N 33/5008; G01N 2500/00; A61P 31/04; G01N 33/542

(57) ABSTRACT

Increasing resistance to antibiotics necessitates discovery of new targets and strategies to combat bacteria. Ideal protein targets are required for viability across many species, are unique to prokaryotes to limit effects on the host and have robust assays to quantitate activity and identify novel inhibitors. Lipoprotein signal peptidase (Lsp) is a transmembrane aspartyl protease required for lipoprotein maturation and entirely fits these criteria. We have developed the first in vitro high-throughput assay to monitor proteolysis by Lsp. We employed our HTS assay against 646,275 compounds to discover inhibitors of Lsp and synthesized a range of analogues to generate molecules with nanomolar IC50 values. Importantly, our inhibitors are effective in preventing the growth of E. coli cultures. Our Lsp assay will be a useful tool for biologists to monitor Lsp activity and our inhibitors will facilitate development of antibacterial agents to potentially treat antibiotic-resistant bacteria.

### CROSS-REFERENCE TO RELATED APPLICATION

This application claims the priority of U.S. provisional application Ser. No. 62/528,759, filed Jul. 5, 2017.

### BACKGROUND

Lipoprotein signal peptidase (Lsp, also known as SPasell) is an aspartic acid protease with a pivotal role in bacterial lipoprotein maturation.^{[1-3]} Lipoproteins have an array of important roles in bacteria that include, but are not limited to, nutrient uptake, adhesion, sporulation, protein transport, secretion, small molecule export, cell wall biosynthesis, and antibiotic resistance.^{[4-6]} During the lipoprotein maturation process, Lsp recognizes a region of the signal sequence within the prolipoprotein termed the lipobox and removes all residues N-terminal to a post-translationally modified diacylglyceryl (DAG)-cysteine residue (FIG. 1A).^{[7]} Lsp is an attractive target for inhibitor discovery for several reasons, including: 1) most bacteria possess a single Isp gene;^{[5]}2) the Lsp sequence and structure is conserved across bacteria; 3) organisms other than bacteria do not have Lsp homologues thus the inhibitor could have high selectivity to bacteria; and 4) the unique mechanism-of-action by Lsp inhibitors should overcome drug-resistant bacteria and may synergistically promote the efficacy of current antibiotics. The x-ray structure of Lsp from Pseudomonas aeruginosa was recently determined and showed that Lsp is a monomer consisting of four transmembrane helices with an active site that lies within the lipid bilayer.^{[8]}

Lsp has been considered as a promising target for the design of new classes of antibiotics for decades, based on the observation that two natural product Lsp inhibitors, globomycin and myxovirescin, have antibacterial efficacy.^{[9-14]} Since their discovery in 1978 from Streptomyces halstedii, the hydrophobic cyclic peptide globomycin and synthetic analogues have demonstrated potent activity against bacterial cultures.^{[9, 15]} A more recent study suggested that another natural product synthesized by Myxococcus xanthus, termed myxovirescin, inhibited Lsp based on the results of a whole cell assay.^{[14, 16]} Unfortunately, the limited stability, in vivo ineffectiveness, scalability, and accessibility currently render these molecules as intractable.^{[17]} Additional Lsp inhibitors with novel mechanisms of action have yet to be identified and the dearth of molecules is primarily due to the lack of a robust and high-throughput in vitro assay.

### SUMMARY

The invention is directed, in various embodiments, to compounds that are effective inhibitors of a bacterial lipoprotein signal peptidase, to methods of identifying such inhibitors using a FRET analysis suitable for high-throughput screening, to methods of inhibiting a bacterial lipoprotein signal peptidase, and to methods of treatment of bacterial infections in patients.

The invention provides, in various embodiments, a compound of formula (1)

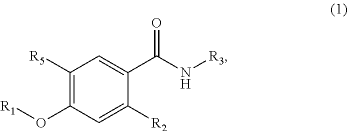

wherein

R₁ is (C1-C6) alkyl, or is a 5-, 6- or 7-membered cycloalkyl;

R₂ is H, NO₂, halo, or trifluoromethyl;

R₃ is a 1,3,4-thiadiazole of formula

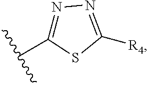

wherein R₄ is (C4-C6) straight or branched chain alkyl, or is a 5-, 6- or 7-membered cycloalkyl;

R₅ is H or (C1-C4)alkoxyl;

or a pharmaceutically acceptable salt thereof.

For example, R₁ can be isopropyl or cyclopentyl. For example, R₂ can be NO₂.

More specifically, the 1,3,4-thiadiazole R₃ can be any one of

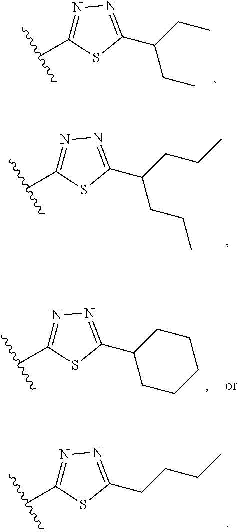

In other embodiments, the invention provides a method of inhibiting a bacterial lipoprotein signal peptidase (Lsp), comprising contacting the peptidase with an effective amount or concentration of a compound of the invention as described herein.

The invention further provides a method of treatment of a bacterial infection in a patient, comprising administering to the patient an effective dose of a compound of the invention, as described herein.

In various embodiments, the invention also provides a method of screening a compound for inhibitory bioactivity of a bacterial lipoprotein signal peptidase (Lsp), comprising contacting a Lsp peptide FRET substrate, comprising a hexapeptide VTGCAK, with a N-terminal dabsyl quencher and C-terminal EDANS fluorophore wherein the cysteine reside of the hexapeptide is S-alkylated with a diacylglycerol residue, and a candidate inhibitor compound, then measuring fluorescence from the fluorophore signalling cleavage of the Lsp FRET substrate and its inhibition by the candidate inhibitor. For example, the screening of multiple compounds can be carried out in parallel in a High Throughput Screening format.

### BRIEF DESCRIPTION OF THE DRAWINGS

FIG. 1. A) Schematic of Lsp cleavage of lipobox residues (green) preceding the DAG-modified cysteine (red). B) Design strategy for a peptide-based FRET reporter substrate with a N-terminal quencher (blue circle) and C-terminal fluorophore (green circle) to rapidly quantitate Lsp activity in vitro.

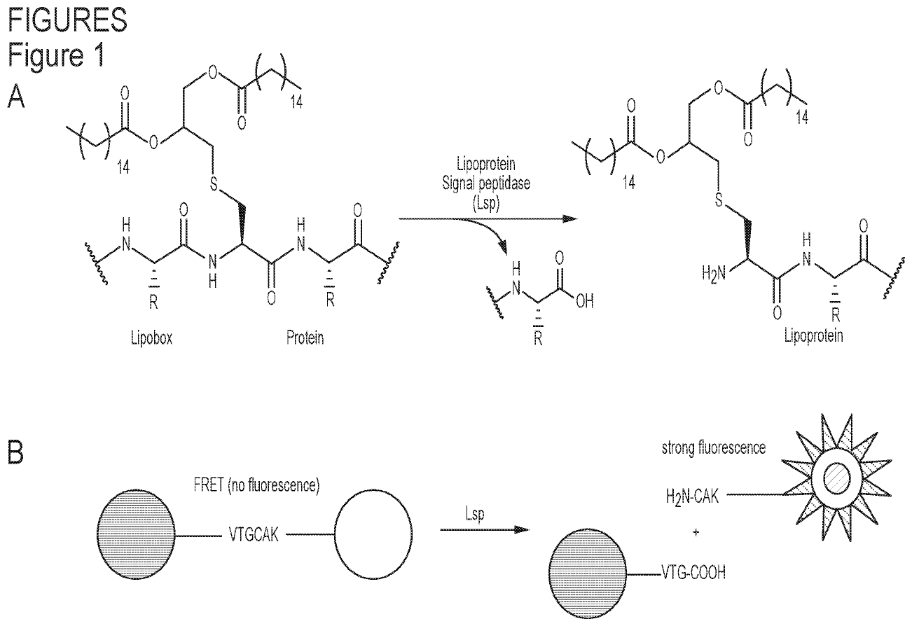

FIG. 2. A) Structure of Lsp peptide FRET substrate with a N-terminal dabsyl quencher (blue) and C-terminal EDANS fluorophore (green). Lsp from E. coli requires the DAG-modified cysteine residue (red) for activity. B). Kinetic assays with Lsp from E. coli. Michaelis-Menton kinetic constants of the optimized FRET substrate are K_{M}=14.2±4.6μM and k_{cat}=0.01±0.0001 s⁻¹. [Lsp]=400 nM.

FIG. 3. In vitro inhibition of E. coli Lsp by 1j (blue), and 1i (green). [S]=50 mM, [Lsp]=100 nM. Mean±SD values are shown. Hill constants are approximately 1 for both compounds, suggesting that there is no cooperativity. Compounds have no effect on cleaved substrate fluorescence.

FIG. 4. Lsp inhibitors in combination with polymyxin B nonapeptide (PMBN) slows the growth of E. coli. Mean±SD values are shown. A) Concentration-response curves against E. coli (ATCC25922) after 16 h incubation. The full time course is shown in FIG. S7. The highest concentration of compound 1j used was 25 mM due to the limited solubility and all assays employed a concentration of PMNB at 8 mg/L. Optical density at 600 nm (OD₆₀₀) values before the bacterial growth (at 0 h) are shown as ‘Background’. B) Lsp-inhibition resistant mutant YX23 is resistant to compound 1j. Bacteria was treated with PMBN (8 mg/L) or 1j (25 mM)+PMBN (8 mg/L). OD₆₀₀ values at 10 h or 24 h are shown for DW37 or YX23, respectively, for comparison between strains. Student's t-test was used to calculate p-values with <0.05 considered significant.

FIG. 5. The effect of linker chain length of 2-6 versus potency is plotted in relation to the compounds of Table 7.

FIG. 6. Concentration-response curves of globomycin on bacteria growth

### DETAILED DESCRIPTION

Herein, we report the design of a robust Lsp substrate and in vitro assay that provides the first rapid and quantitative method to study Lsp biochemistry and serves as a platform to discover small-molecule inhibitors. See: S. Kitamura, A. Owensby, D. Wall, D. W. Wolan, Cell Chemical Biology 2017, 25, 301-308.e312. (https://www.cell.com/cell-chemical-biology/fulltext/S2451-9456(17)30460-9) and Kitamura, S. and Wolan, D. W. (2018) FEBS Lett. .doi:10.1002/1873-3468.13155 (https://febs.onlinelibrary.wiley.com/doi/abs/10.1002/1873-3468.13155)

We optimized our assay for ultra-high-throughput discovery approaches and screened an in-house library of 646,275 compounds for small molecule inhibitors of Lsp. From this effort, several lead inhibitors were identified, and further medicinal chemistry efforts led to the development of compound 1j with an IC₅₀ of 99 nM. We subjected 1j to cultures of E. coli and demonstrated that the small molecule inhibits bacterial growth.

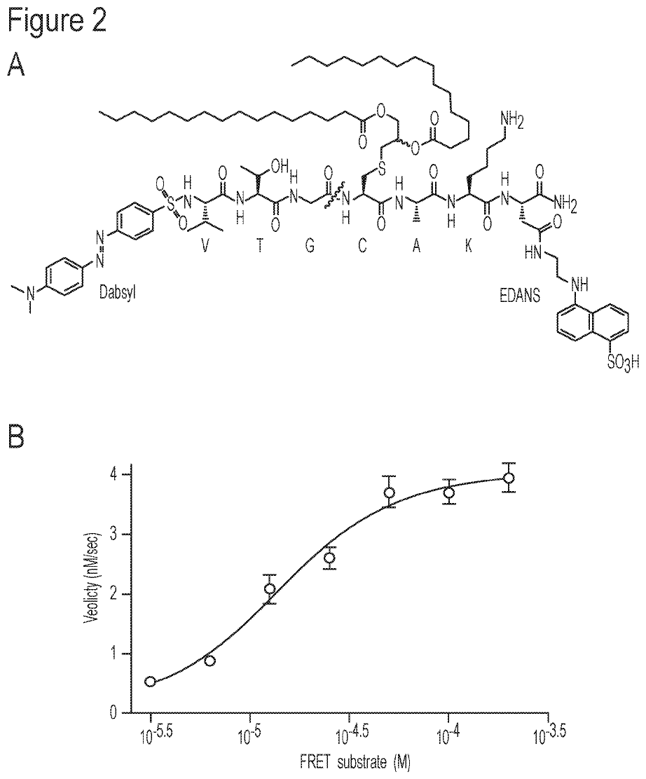

To interrogate and measure exogenously purified E. coli Lsp activity, we synthesized FRET-based peptide substrates consisting of a N-terminal dabsyl quencher and a C-terminal EDANS fluorophore by Fmoc-based solid-phase peptide synthesis (SPPS) (FIG. 2A). We found that peptide elongation with DAG-Cys required low-loading capacity resin, and the purification of the peptide required normal phase HPLC using an amide column (detailed in the Supporting Information). Consistent with previous reports,^{[1]} a DAG-Cys modification is essential for substrate recognition in our in vitro conditions, as E. coli Lsp was unable to hydrolyze FRET substrates that lacked a lipid tail (data not shown). After optimizing the peptide sequence, DAG structure, and fluorophore pairs, our Lsp substrate contains the peptide sequence -VTG/CAK- and a DAG-modified cysteine with saturated palmitate fatty acids as shown in FIG. 2A. Michaelis-Menten kinetic measurements were performed for the hydrolysis of the FRET substrate by Lsp from E. coli and has a K_{M}=14.2±4.6 μM and k_{cat}=0.01±0.0001 s⁻¹ (FIG. 2B).

We subsequently validated our optimized assay by determining the inhibitory potency of globomycin. Globomycin inhibited Lsp from E. coli with IC₅₀<5 nM with an enzyme concentration at 10 nM (FIG. S1).^{[3, 20]} This is consistent with previous reports that globomycin is a potent inhibitor of Lsp.^{[3, 20]} In addition, myxovirescin B showed comparable inhibitory potency to globomycin with an IC₅₀<5 nM (FIG. S1). As previously proposed,^{[2]} no known aspartic acid protease inhibitors, including general (pepstatin), γ-secretase (begacestat and semagacestat), renin (aliskiren), and HIV inhibitors (saquinavir, atazaniavir) inhibit E. coli Lsp, despite the active site consisting of the canonical two asparatic acid residues responsible for the activation of a water molecule for nucleophilic attack on the substrate peptide backbone carbonyl.[8] To identify new small molecule Lsp inhibitors we therefore performed a high-throughput screen against an in-house compound library.

The Lsp FRET assay was miniaturized to 1536-well plates and ultra-high-throughput screening was performed against 646,275 molecules from the Scripps Drug Discovery Library. After eliminating molecules that interfere the fluorescence signal and molecules considered “PAINS” due to promiscuous reactivity,^{[21-23]} we identified several compounds of interest for further study. Based on the potency and synthetic accessibility, a benzamide compound 1a was chosen for structural optimization. Biochemical analysis revealed that 1a inhibits Lsp in non-competitive manner. Unlike previous attempts at structure-activity relationships (SAR) with globomycin and myxovirescin, relying on visualization of Braun's lipoprotein modification by gel-shift assays or MIC determination,^{[14, 15, 24]} we were able to perform rapid iterative rounds of synthesis and inhibition evaluation with our in vitro assay.

The following Tables 1-7 provide structure-activity relationships for various compounds useful for practice of a method of the invention.

TABLE 1
Structure-activity relationships of benzamide 1a.

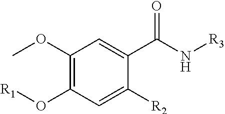

| | | | | IC₅₀^{a} |
|---|---|---|---|---|
| # | R₁ | R₂ | R₃ | (μM) |
| 1a | CH₃ | NO₂ | 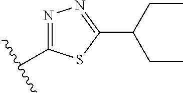 [SMILES: *c1nnc(C(CC)CC)s1] | 4.2 (±1.0)^{b} |
| 1b | CH₃ | H | 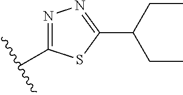 [SMILES: *c1nnc(C(CC)CC)s1] | 110 (±23)^{b} |
| 1c | CH₃ | NO₂ | 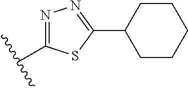 [SMILES: *c1nnc(C2CCCCC2)s1] | >400^{b} |
| 1d | CH₃ | NO₂ | 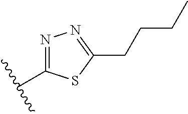 [SMILES: *c1nnc(CCCC)s1] | 29 (±0.60)^{b} |
| 1e | CH₃ | NO₂ | 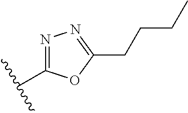 [SMILES: *c1nnc(CCCC)o1] | >400^{b} |
| 1f | CH₃CH₂ | NO₂ | 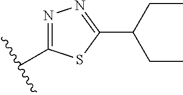 [SMILES: *c1nnc(C(CC)CC)s1] | 1.2 (±0.10)^{b} |
| 1g | 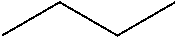 | NO₂ | 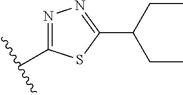 [SMILES: *c1nnc(C(CC)CC)s1] | 0.54 (±0.15)^{c} |
| 1h | 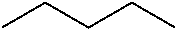 | NO₂ | 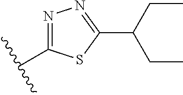 [SMILES: *c1nnc(C(CC)CC)s1] | 0.11 (±0.025)^{c} |
| 1i | 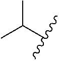 [SMILES: *C(C)C] | NO₂ | 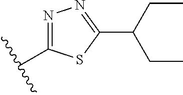 [SMILES: *c1nnc(C(CC)CC)s1] | 0.21 (±0.036)^{c} |
| 1j | 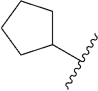 [SMILES: *C1CCCC1] | NO₂ | 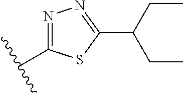 [SMILES: *c1nnc(C(CC)CC)s1] | 0.099 (±0.022) ^{c} |

^{a}IC₅₀ values were determined using a FRET-based assay against Lsp from E. coli.
Mean (± SD) values are shown.
^{b}[Lsp] = 400 nM.
^{c}[Lsp] = 100 nM.

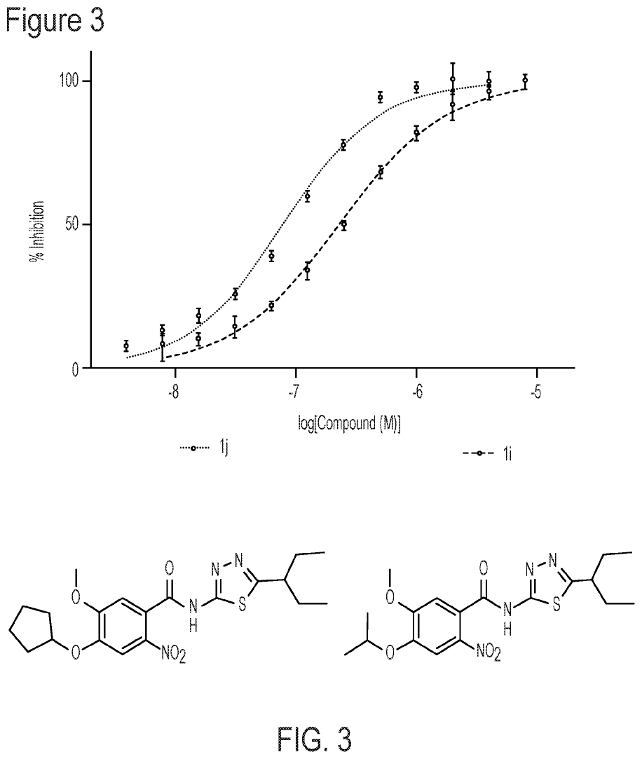

SAR of key analogues of 1a are shown in Table 1 with comprehensive SAR lists shown in Tables S2-S4. Briefly, removal of NO₂ (1b) as well as restricting the conformation of R₃ with a cyclohexyl (1c) ablated the potency. Replacement of thiadiazole (1d) with oxadiazole led to an inactive compound (1e), indicating the importance of sulfur in the thiadiazole moiety. Elongation at R₁ from methyl to ethyl (1f) improved the potency to 1.2 μM. Further elongation (1g, 1h, 1i) as well as introduction of cyclopentyl moiety (1j) on R₁ leads to the improvement of the potency up to 0.1 μM. We observed a positive correlation between clogP (calculated using ChemDraw 16.0) and pIC₅₀ in compound series that have different alkyl groups on R₁ position. This correlative relationship assisted in our design and selection for more potent inhibitors. Based on the inhibitory potency and relatively low lipophilicity (1i: clogP=3.1, 1j: clogP=3.6), we chose compounds 1i and 1j for further biological characterization. Concentration-inhibition curves of compounds 1i and 1j are shown in FIG. 3.

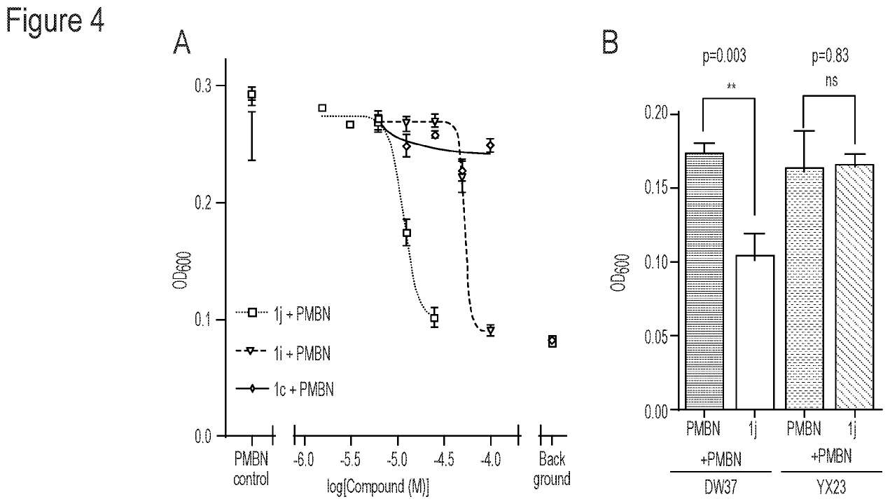

As a proof of concept, we tested the effects of these inhibitors on bacterial growth of E. coli (FIG. 4). Although these compounds alone did not inhibit the growth of E. coli, Lsp inhibitors in combination with polymyxin B nonapeptide (PMBN), which improves the outer-membrane permeability of compounds,^{[25]} slowed the growth of bacteria in a concentration-dependent manner (FIG. 4A). Compound 1j showed almost 100% growth inhibition at 25 μM (MIC=25 μM=11 μg/mL under this condition), while compound 1i required a concentration of 100 μM to block growth (MIC=100 μM=41 μg/mL). Compound 1c, which has IC₅₀>400 μM in the enzyme assay (Table 1), did not inhibit the bacterial growth, suggesting that these effects are Lsp-inhibition dependent.

To further validate that the effects on the growth of E. coli are directly due to the inhibition of Lsp, we next measured the effects of 1j on a diagnostic E. coli strain, YX23. YX23 is a mutant E. coli that has an IS4 insertion in the gene that encodes Braun's lipoprotein, and was shown to be resistant to both myxovirescin and globomycin.^{[14]} As shown in FIG. 4B, compound 1j inhibited the growth of the isogenic parent strain (DW37) while it did not inhibit the growth of YX23. These data strongly suggest that the growth inhibitory effects of compound 1j is specific toward Lsp.

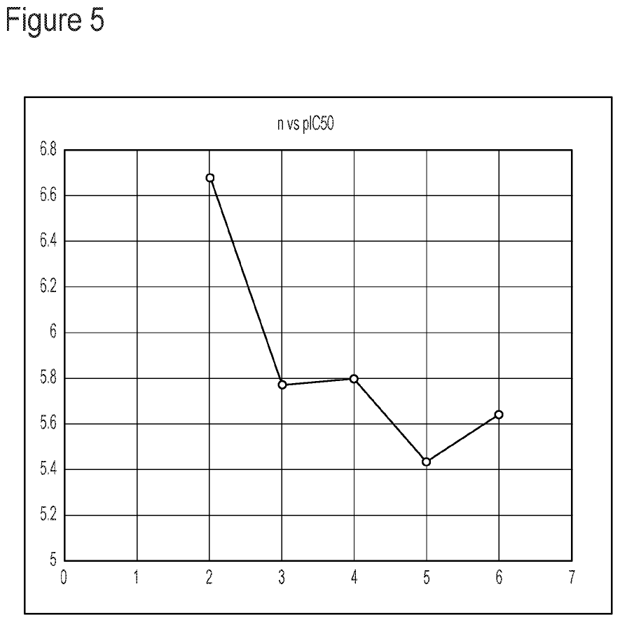

Further data relating to structure-activity relationships are shown in Tables 2-7, and FIG. 5, below.

TABLE 2

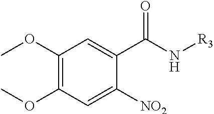

| | | IC₅₀ |
|---|---|---|
| # | R₃ | (μM) |
| Benzamide SR270728 | 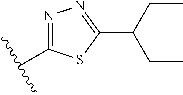 [SMILES: *c1nnc(C(CC)CC)s1] | 4.2 ± 1.0 |
| Sk061-69-2 | 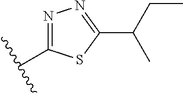 [SMILES: *c1nnc(C(C)CC)s1] | 8.7 |
| Sk061-69-1 | 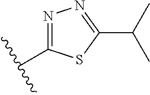 [SMILES: *c1nnc(C(C)C)s1] | 44 |
| Sk061-69-3 | 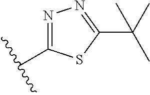 [SMILES: *c1nnc(C(C)(C)C)s1] | 36 |
| Sk054-141-2 | 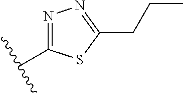 [SMILES: *c1nnc(CCC)s1] | 26 |
| 7960275 | 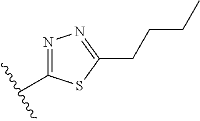 [SMILES: *c1nnc(CCCC)s1] | 29 ± 0.60 |
| Sk052-147-2 | 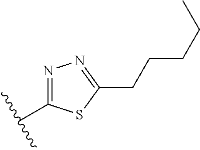 [SMILES: *c1nnc(CCCCC)s1] | 26 |
| Sk061-69-4 | 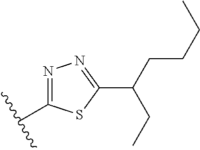 [SMILES: *c1nnc(C(CC)CCCC)s1] | 2.5 |
| Sk052-88-2 | 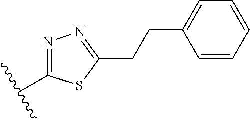 [SMILES: *c1nnc(CCc2ccccc2)s1] | 20 |
| Sk052-147-1 | 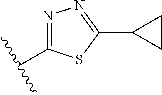 [SMILES: *c1nnc(C2CC2)s1] | 208 |
| Sk052-56 | 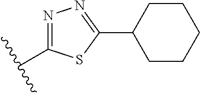 [SMILES: *c1nnc(C2CCCCC2)s1] | >400 |
| Sk052-88-1 | 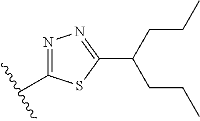 [SMILES: *c1nnc(C(CCC)CCC)s1] | 1.1 |
| Sk052-27-2 | 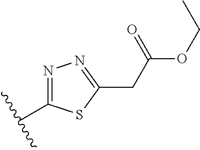 [SMILES: *c1nnc(CC(=O)OCC)s1] | 78 |
| Sk052-147-3 | 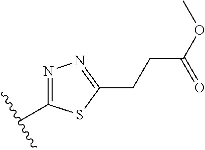 [SMILES: *c1nnc(CCC(=O)OC)s1] | >200 |

TABLE 3

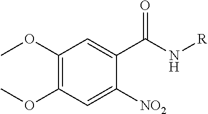

| | | IC₅₀ |
|---|---|---|
| # | R | (μM) |
| Sk061-69-1 | 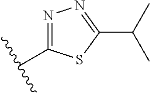 [SMILES: *c1nnc(C(C)C)s1] | 44 |
| Sk056-82-3 | 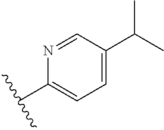 [SMILES: *c1ccc(C(C)C)cn1] | >400 |
| Sk056-82-4 | 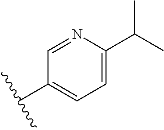 [SMILES: *c1ccc(C(C)C)nc1] | >400 |

TABLE 4

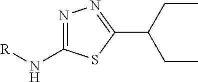

| | | IC₅₀ |
|---|---|---|
| # | R | (μM) |
| Sk052-146-2 | 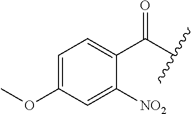 [SMILES: *C(=O)c1ccc(OC)cc1[N+](=O)[O-]] | 2.2 |
| Sk052-146-1 | 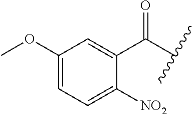 [SMILES: *C(=O)c1cc(OC)ccc1[N+](=O)[O-]] | 3.6 |
| Sk054-91-2 | 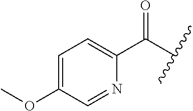 [SMILES: *C(=O)c1ccc(OC)cn1] | >400 |
| Sk054-91-3 | 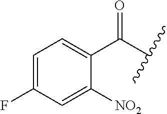 [SMILES: *C(=O)c1ccc(F)cc1[N+](=O)[O-]] | 7.2 |
| Z123635394 | 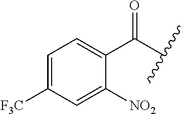 [SMILES: *C(=O)c1ccc(C(F)(F)F)cc1[N+](=O)[O-]] | 9.3 |
| Sk054-112-2 | 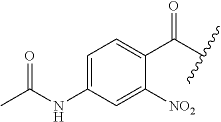 [SMILES: *C(=O)c1ccc(NC(C)=O)cc1[N+](=O)[O-]] | 160 |
| Sk054-142 | 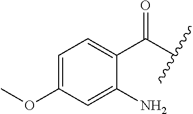 [SMILES: *C(=O)c1ccc(OC)cc1N] | 11 |
| Sk056-45-1 | 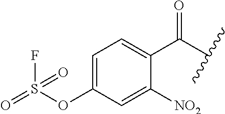 [SMILES: *C(=O)c1ccc(OS(=O)(=O)F)cc1[N+](=O)[O-]] | 46 |
| Sk056-50-4 | 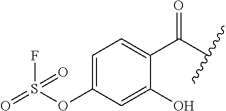 [SMILES: *C(=O)c1ccc(OS(=O)(=O)F)cc1O] | 290 |
| Sk056-45-3 | 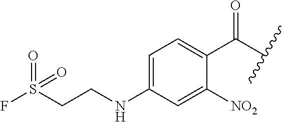 [SMILES: *C(=O)c1ccc(NCCS(=O)(=O)F)cc1[N+](=O)[O-]] | 19 |
| Sk054-144 | 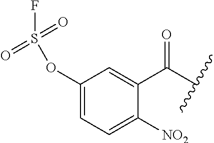 [SMILES: *C(=O)c1cc(OS(=O)(=O)F)ccc1[N+](=O)[O-]] | 65 |
| Sk054-141-1 | 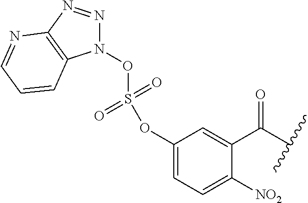 [SMILES: *C(=O)c1cc(OS(=O)(=O)On2nnc3ncccc32)ccc1[N+](=O)[O-]] | 120 |
| Sk054-91-1 | 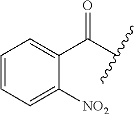 [SMILES: *C(=O)c1ccccc1[N+](=O)[O-]] | 11 ± 3 |
| Sk056-82-2 | 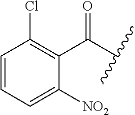 [SMILES: *C(=O)c1c(Cl)cccc1[N+](=O)[O-]] | 260 |
| Sk056-24 | 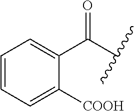 [SMILES: *C(=O)c1ccccc1C(=O)O] | 270 |
| Sk056-13-2 | 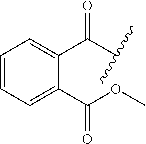 [SMILES: *C(=O)c1ccccc1C(=O)OC] | 12 |
| Sk056-82-1 | 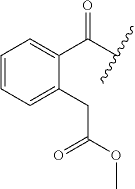 [SMILES: *C(=O)c1ccccc1CC(=O)OC] | 21 |

TABLE 5

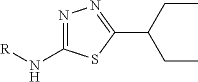

| | | IC₅₀ |
|---|---|---|
| # | R | (nM) |
| Sk054-116-1 | 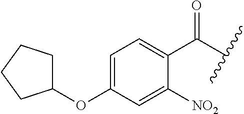 [SMILES: *C(=O)c1ccc(OC2CCCC2)cc1[N+](=O)[O-]] | 400 |
| Sk052-145-2 | 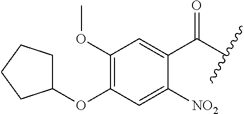 [SMILES: *C(=O)c1cc(OC)c(OC2CCCC2)cc1[N+](=O)[O-]] | 73 |
| Sk054-82-3 | 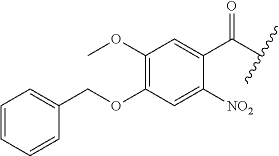 [SMILES: *C(=O)c1cc(OC)c(OCc2ccccc2)cc1[N+](=O)[O-]] | 2900 |
| Sk054-116-2 | 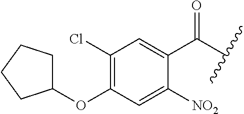 [SMILES: *C(=O)c1cc(Cl)c(OC2CCCC2)cc1[N+](=O)[O-]] | 110 ± 12 |
| Sk056-22-1 | 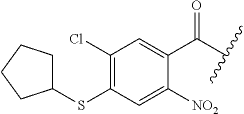 [SMILES: *C(=O)c1cc(Cl)c(SC2CCCC2)cc1[N+](=O)[O-]] | 20 |
| Sk056-16-1 | 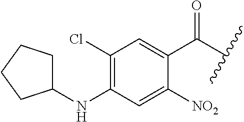 [SMILES: *C(=O)c1cc(Cl)c(NC2CCCC2)cc1[N+](=O)[O-]] | 170 |
| Sk056-29 | 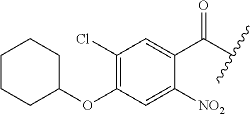 [SMILES: *C(=O)c1cc(Cl)c(OC2CCCCC2)cc1[N+](=O)[O-]] | 68 |
| Sk061-31-1 | 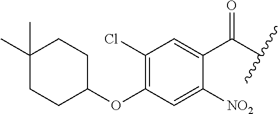 [SMILES: *C(=O)c1cc(Cl)c(OC2CCC(C)(C)CC2)cc1[N+](=O)[O-]] | 230 E = 100 nM |
| Sk061-31-2 | 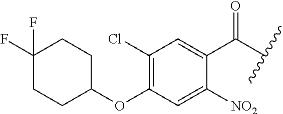 [SMILES: *C(=O)c1cc(Cl)c(OC2CCC(F)(F)CC2)cc1[N+](=O)[O-]] | 580 E = 100 nM |
| Sk056-22-2 | 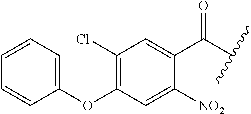 [SMILES: *C(=O)c1cc(Cl)c(Oc2ccccc2)cc1[N+](=O)[O-]] | 190 |
| Sk061-30-1 70% pure | 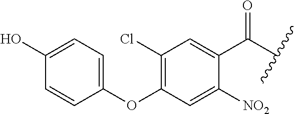 [SMILES: *C(=O)c1cc(Cl)c(Oc2ccc(O)cc2)cc1[N+](=O)[O-]] | >4,000 E = 100 nM |
| Sk061-32 | 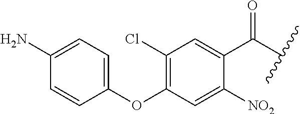 [SMILES: *C(=O)c1cc(Cl)c(Oc2ccc(N)cc2)cc1[N+](=O)[O-]] | ~4,000 E = 100 nM |
| Sk061-29 | 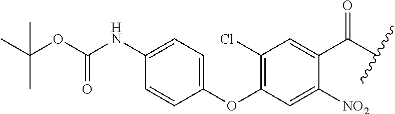 [SMILES: *C(=O)c1cc(Cl)c(Oc2ccc(NC(=O)OC(C)(C)C)cc2)cc1[N+](=O)[O-]] | 640 E = 100 nM |
| Sk061-45 | 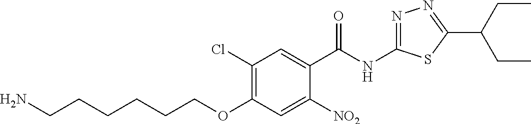 | >4,000 E = 100 nM |
| Sk061-54 | 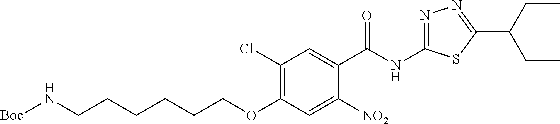 | ~2300 E = 100 nM |

[Lsp] = 20 nM

TABLE 6

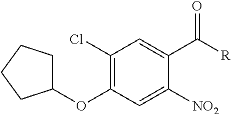

| | | IC₅₀ |
|---|---|---|
| # | R | (nM) |
| Sk054-116-2 | 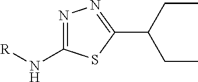 | 110 ± 12 |
| Sk056-15 | 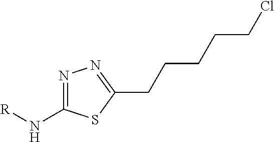 | 3,800 |
| Sk056-26 | 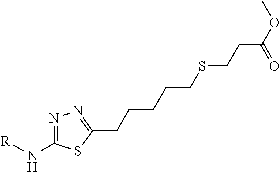 | 3,200 |
| Sk056-27 | 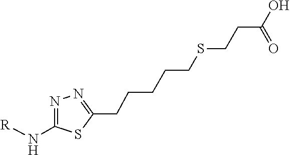 | 11,000 |

TABLE 7

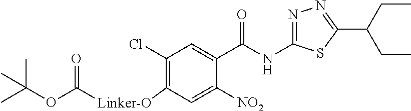

| | | | IC₅₀^{a} |
|---|---|---|---|
| | | | (μM) |
| # | Linker | | E = 100 nM |
| Sk061-68 | 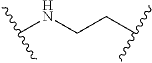 [SMILES: *CCN*] | 2 (ethyl) | 0.21 (0.53) |
| Sk061-80-1 | 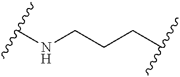 [SMILES: *CCCN*] | 3 (propyl) | 1.7 |
| Sk061-80-2 | 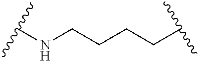 [SMILES: *CCCCN*] | 4 (butyl) | 1.6 |
| Sk061-80-3 | 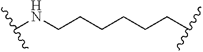 [SMILES: *CCCCCCN*] | 5 (pentyl) | 3.7 |
| Sk061-54 | 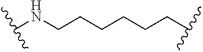 [SMILES: *CCCCCCN*] | 6 (hexyl) | 2.3 |
| Sk061-29 | 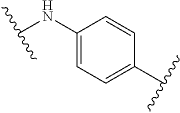 [SMILES: *Nc1ccc(*)cc1] | p-Phe | 0.64 |
| Sk061-80-4 | 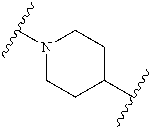 [SMILES: *C1CCN(*)CC1] | Piperidine | 1.1 |
| Sk061-80-5 | 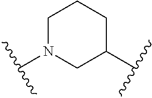 [SMILES: *C1CCCN(*)C1] | piperidine | 0.83 |

The effect of linker chain length from 2-6 carbon atoms (ethyl-hexyl) on IC50 is shown in FIG. 5.

In summary, we have developed a high-throughput assay to monitor Lsp activity and have employed an HTS and medicinal chemistry campaign using this assay to look for Lsp inhibitors. Our study emphasizes the strength of combining chemical biology, HTS, and SAR for the development of chemical probes in microbiology and bacteriology as well as novel antibacterial candidates. The general SPPS method used to synthesize the Lsp FRET substrate can be rapidly optimized for lipid lengths and saturation, peptide sequence and length, and FRET pairs for any bacterial Lsp enzyme (FIG. 2A). Importantly, we discovered novel small molecule E. coli Lsp inhibitors with our HTS assay. Structural optimization from a hit compound 1a led to the generation of a class of molecules with nanomolar IC₅₀ values in vitro as well as growth inhibitory properties against E. coli. These compounds will be ideal leads for further medicinal chemistry to improve the potency, permeability, solubility and selectivity, especially with our rapid assay and an x-ray structure of Lsp available.^{[8]} Although high-throughput screening and target-based approach has been challenging for antibiotic development,^{[26, 27]} our work demonstrates the utility of HTS in the discovery of antimicrobial leads, as an alternative approach for traditional natural product-based approaches.^{[28, 29]} Our assay will stimulate the study of Lsp and bacterial lipoprotein maturation pathways, and our inhibitors further validate Lsp as an exciting new target for development of antibiotics.

### DOCUMENTS CITED

[1] N. Buddelmeijer, FEMS Microbiol. Rev. 2015, 39, 246-261.
[2] M. Paetzel, A. Karla, N. Strynadka, Chem. Rev. 2002, 102, 4549-4580.
[3] M. Tokunaga, J. M. Loranger, H. C. Wu, J. Biol. Chem. 1984, 259, 3825-3830.
[4] M. M. Babu, M. L. Priya, A. T. Selvan, M. Madera, J. Gough, L. Aravind, K. Sankaran, J. Bacteriol. 2006, 188, 2761-2773.
[5] A. Kovacs-Simon, R. W. Titball, S. L. Michell, Infect. Immun. 2011, 79, 548-561.
[6] I. C. Sutcliffe, R. R. Russell, J. Bacteriol. 1995, 177, 1123-1128.
[7] M. T. N. Nguyen, T. Van Kersavond, S. H. L. Verhelst, ACS Chem. Biol. 2015, 10, 2423-2434.
[8] L. Vogeley, T. El Arnaout, J. Bailey, P. J. Stansfeld, C. Boland, M. Caffrey, Science 2016, 351, 876-880.
[9] M. Inukai, R. Enokita, A. Torikata, M. Nakahara, S. Iwado, M. Arai, J. Antibiot. 1978, 31, 410-420.
[10] M. Inukai, M. Takeuchi, K. Shimizu, M. Arai, J. Antibiot. 1978, 31, 1203-1205.
[11] K. Gerth, H. Irschik, H. Reichenbach, W. TROWITZSCH, J. Antibiot. 1982, 35, 1454-1459.
[12] H. Kogen, T. Kiho, M. Nakayama, J. Am. Chem. Soc. 2000, 122, 10214-10215.
[13] D. R. Williams, J. M. McGill, J. Org. Chem. 1990, 55, 3457-3459.
[14] Y. Xiao, K. Gerth, R. Miller, D. Wall, Antimicrob. Agents Chemother. 2012, 56, 2014-2021.
[15] T. Kiho, M. Nakayama, K. Yasuda, S. Miyakoshi, M. Inukai, H. Kogen, Bioorg. Med. Chem. Lett. 2003, 13, 2315-2318.
[16] Y. Paitan, E. Orr, E. Z. Ron, E. Rosenberg, J. Bacteriol. 1999, 181, 5644-5651.
[17] T. Kiho, H. Kogen, J. Synth. Org. Chem. 2005, 1, 51-62.
[18] Y. Xiao, D. Wall, J. Bacteriol. 2014, 196, 1174-1183.
[19] I. K. Dev, P. H. Ray, J. Biol. Chem. 1984, 259, 11114-11120.
[20] I. K. Dev, R. J. Harvey, P. H. Ray, J. Biol. Chem. 1985, 260, 5891-5894.
[21] M. Pouliot, S. Jeanmart, J. Med. Chem. 2016, 59, 497-503.
[22] J. B. Baell, G. A. Holloway, J. Med. Chem. 2010, 53, 2719-2740.
[23] J. L. Dahlin, J. W. M. Nissink, J. M. Strasser, S. Francis, L. Higgins, H. Zhou, Z. Zhang, M. A. Walters, J. Med. Chem. 2015, 58, 2091-2113.
[24] T. Kiho, M. Nakayama, K. Yasuda, S. Miyakoshi, M. Inukai, H. Kogen, Bioorg. Med. Chem. 2004, 12, 337-361.
[25] M. Vaara, Microbiol. Rev. 1992, 56, 395-411.
[26] D. J. Payne, M. N. Gwynn, D. J. Holmes, D. L. Pompliano, Nat. Rev. Drug Discov. 2007, 6, 29-40.
[27] R. Tommasi, D. G. Brown, G. K. Walkup, J. I. Manchester, A. A. Miller, Nat. Rev. Drug Discov. 2015, 14, 529-542.
[28] G. D. Wright, Can. J. Microbiol. 2014, 60, 147-154.
[29] C. T. Walsh, T. A. Wencewicz, J. Antibiot. 2014, 67, 7-22.

### EXAMPLES

### Experimental Section

General.

All reagents and solvents were purchased from commercial suppliers and were used without further purification. Globomycin and polymyxin B nonapeptide hydrochloride, cationic cyclic peptide (PMBN) were purchased from Sigma-Aldrich (purity≥98%). Compounds 1b and 1d were purchased from Enamine Ltd (purity≥90%) or from ChemBridge, respectively. All reactions were performed in an inert atmosphere of dry nitrogen or argon. ¹H and ¹³C NMR spectra were collected using a Bruker 600, 500, or 400 MHz spectrometer with chemical shifts reported relative to residual deuterated solvent peaks or a tetramethylsilane internal standard. Accurate masses were measured using an ESI-TOF (HRMS, Agilent MSD) or MSQ Plus mass spectrometer (LRMS, Thermo Scientific). Reactions were monitored on TLC plates (silica gel 60, F254 coating, EMD Millipore, 1057150001), and spots were either monitored under UV light (254 mm) or stained with phosphomolybdic acid. The same TLC system was used to test purity, and all final products showed a single spot on TLC with both phosphomolybdic acid and UV absorbance. The purity of the compounds that were tested in the assay was >95% based on ¹H NMR and reverse phase HPLC-UV on monitoring absorption at 254 nm. It should be noted that Lsp is susceptible to divalent cations such as Cu²⁺, Zn²⁺, thus care was taken to ensure that the final products did not contain contaminations of these metals.

### Analytical LC Method to Determine the Purity of Synthetic Compounds

Purity determination of synthetic compounds was performed on a Thermo Scientific Accela HPLC system using Accela 1250 pump. The Thermo Accucore C18 RP HPLC column (150 mm×2.1 mm, particle size 2.6 μm) was used. The UV absorption between 190 nm and 400 nm was monitored, and the purity was determined by the peak area at 254 nm. Solvent used were Milli-Q water 99.9/Formic acid 0.1, versus Acetonitrile 99.9/Formic acid 0.1.

Expression and Purification of Lsp from E. coli.

The full-length Lsp clone from E. coli (residues 1-164) was generated using standard PCR-based cloning and verified via double-stranded plasmid sequencing. Lsp is over-expressed as a N-terminal His₆-tag fusion with an entrokinase cleavage site from E. coli Lemo21 (DE3) (New England Biosciences) in a pET19b vector (Novagen). Cells were grown in 2xYT media supplemented with 50 μg/ml carbenicillin at 37° C. to an OD₆₀₀ of 0.6-0.8. Flasks were then transferred to 16° C. and protein expression was induced with 0.1 mM IPTG for 16 h. Cells were immediately harvested and resuspended in ice cold PBS, pH 7.4, 5% glycerol, 14 mM 2-mercaptoethanol (BME) (buffer A) supplemented with a Roche Complete inhibitor tablet (per 50 mL of buffer) and subjected to 3 cycles of lysis by microfluidization (Microfluidics). The cellular debris was removed by centrifugation at 24,000×g for 15 min at 4° C. and the membrane fraction was isolated by subsequent ultracentrifugation at 100,000×g for 1 hr at 4° C. Membrane pellets was resuspended in buffer A containing 1% n-dodecyl-β-D-maltopyranoside (DDM) and stirred gently for 4 hr at 4° C. to solubilize the membrane. The membrane solutions were subjected to another round of ultracentrifugation at 100,000×g for 45 min at 4° C. to remove non-soluble contents. A final concentration of 20 mM imidazole was added to the supernatant along with 5 ml of Ni-NTA bead slurry for 1 hr at 4° C. Beads were washed 10 CVs of PBS, pH 7.4, 5% glycerol, 10 mM BME, 0.1% DDM (buffer B) and 50 mM imidazole and eluted with buffer B containing 250 mM imidazole. The eluted protein was concentrated down to 5 mL using Millipore Ultrafree-15 devices with a MWCO of 50,000 Da and subjected to gel filtration chromatography (Superdex 200, GE Amersham) in buffer B. Fractions containing Lsp were pooled, concentrated, and immediately stored at −70° C. Pure Lsp yields are approximately 1-5 mg/L of culture with >95% purity, as assessed by SDS-PAGE.

Lsp FRET Substrate Synthesis.

The Lsp peptide FRET substrate was designed based on known lipobox sequences and synthesized using standard Fmoc solid-phase synthesis chemistry starting with the coupling of Fmoc-Asp(EDANS)-OH (EMD Millipore) to TGR low-capacity resin (Novasyn). After completion of the peptide synthesis and the N-terminal capping with dabsyl chloride, the substrate was released from the resin with a cocktail of trifluoroacetic acid (TFA), triisopropylsilane (TIPS), and water (95%:2.5%:2.5%). Crude peptide was purified by normal-phase HPLC using an XBridge™ Prep Amide column (Waters, 5 μm, 19×100 mm) with a 10-100% MeOH/DCM gradient as shown in the table below. The final purity of Lsp FRET substrate exceeded 95% purity on LC-UV (254 nm, figure below) and the structure was verified by HRMS (m/z Calcd. for (M+H+) 1777.9705. Found 1777.9708).

### Lsp Activity Assay, Michaelis-Menten Kinetics, and IC₅₀ Determination.

Michaelis-Menten kinetic analysis was performed with 400 nM of the wild type E. coli Lsp in a buffer consisting of PBS, pH 7.4, 5% glycerol, 0.5% DDM. To determine IC₅₀ values, Lsp was incubated at 10-400 nM in the presence of increasing amounts of inhibitor in a reaction buffer consisting of PBS, pH 7.4, 5% glycerol, 0.5% DDM, and 2.5% DMSO, and incubated for 30 min at 25° C. 50 μM Lsp FRET substrate was subsequently added and the rate of substrate hydrolysis was measured by increase in fluorescence (excitation 355 nm, emission 495 nm) in 96-well plates on a PerkinElmer EnVision plate reader or Tecan Safire 2 microplate reader. Michaelis-Menten constants and IC₀ values were determined using GraphPad Prism software (GraphPad, Inc.).

High-Throughput Screening.

The HTS Lsp inhibitor assay protocol was modified based on optimized fluorescence that resulted from cleavage of Lsp FRET substrate by 200 nM E. coli Lsp in an activity buffer consisting of PBS, pH 7.4, 5% glycerol, 0.5% DDM, 6% DMSO and 10 mM DTT. The 96-well assay volume was miniaturized to 1536-well plates and performed in a total volume of 5 μL with 200 nM E. coli Lsp incubated with 8.4 μM compound (646,275 molecules total from the Scripps Drug Discovery Library) for 30 min at 25° C. prior to adding the FRET substrate to 20 μM. After a 60-min incubation, Lsp activity was quenched with 83 μM ZnCl₂ and the fluorescence was read on an EnVision plate reader (excitation 355 nm, emission 495 nm). The assay was well behaved, as evidenced by a Z′ of 0.69±0.05, signal-to-background (S:B) of 1.35±0.05, hit cutoff of 27.9% inhibition, and 2271 inhibitors (0.35% hit rate). Globomycin and the DMSO vehicle served as the positive and negative controls, respectively, and all active compounds were re-screened in triplicate for verification as well as assessed for false-positive features against a counterscreen (e.g., compounds added after quenching Lsp activity) with 344 molecules demonstrating selective Lsp activity (Z′=0.74±0.02, S:B=1.41±0.01). Subsequent 10-point titration curves identified 17 compounds with IC₅₀<5 μM with no response in the counterscreen conditions (Z′=0.73±0.03, S:B=1.32±0.01). Several molecules were eliminated from further study due to having known promiscuous reactive groups (i.e., “PAINS”). Effect of DMSO concentration on Lsp activity is shown below ([Lsp]=100 nM in 96 well plate format).

Bacteria Growth Assay.

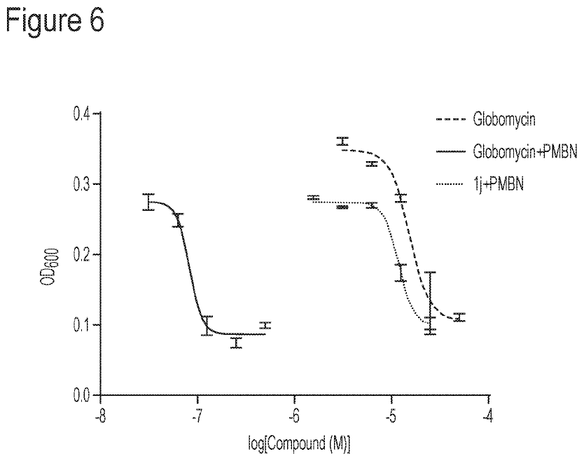

Effect of compounds on bacterial growth was measured using the method described previously with slight modifications^{[1]. Colony suspension method was used to prepare bacteria suspension using McFarland Standard} 0.5 to adjust the turbidity. Cells were introduced to two-fold serial dilutions of compound in a final volume of 105 μL with 5% DMSO. Mueller-Hinton broth (MH) or LB broth was used for E. coli ATCC25922 or E. coli DW37 and YX23, respectively. Plates were incubated at 37° C., and the OD₆₀₀ was measured using a PerkinElmer EnVision plate reader after gentle shaking. Mutant E. coli (DW37 and YX23) were previously described.^{[2]} Globomycin was used as a positive control and the concentration-response curves of globomycin on bacteria growth (E. coli ATCC25922) is shown below (error bar: SD). See FIG. 6.

### In Vitro Stability Measurements.

### Microsomal Stability:

Incubations were carried out in 96-well plates in 5 aliquots of 40 μL each (one for each time point) in duplicates. Liver microsomal incubation medium contained PBS (100 mM, pH 7.4), MgCl₂ (3.3 mM), NADPH (3 mM), glucose-6-phosphate (5.3 mM), glucose-6-phosphate dehydrogenase (0.67 units/mL) with 0.42 mg of human liver microsomal protein per mL. Control incubations were performed replacing the NADPH-cofactor system with PBS. Test compound (2 μM, final solvent concentration 1.6%) was incubated with microsomes at 37° C., shaking at 100 rpm. Five time points over 40 minutes had been analyzed. The reactions were stopped by adding 12 volumes of 90% acetonitrile-water to incubation aliquots, followed by protein sedimentation by centrifuging at 5500 rpm for 3 minutes. Supernatants were analyzed using the HPLC system coupled with tandem mass spectrometer using Shimadzu VP HPLC system including vacuum degasser, gradient pumps, reverse phase HPLC column, column oven and autosampler. The HPLC system was coupled with tandem mass spectrometer API 3000 (PE Sciex). The TurbolonSpray ion source was used in both positive and negative ion modes. Acquisition and analysis of the data were performed using Analyst 1.5.2 software (PE Sciex).

### Plasma Stability:

Incubations were carried out in 5 aliquots of 70 μL each (one for each time point), in duplicates. Test compounds (1 μM, final DMSO concentration 1%) were incubated at 37° C. with shaking at 100 rpm. Five time points over 120 minutes have been analyzed. The reactions were stopped by adding 420 μL of acetonitrile-water mixture (90:10) with subsequent plasma proteins sedimentation by centrifuging at 5500 rpm for 5 minutes. Supernatants were analyzed by the same HPLC system to the microsomal stability assay. The percentage of the test compounds remaining after incubation in plasma and their half-lives (T_{1/2}) were calculated.

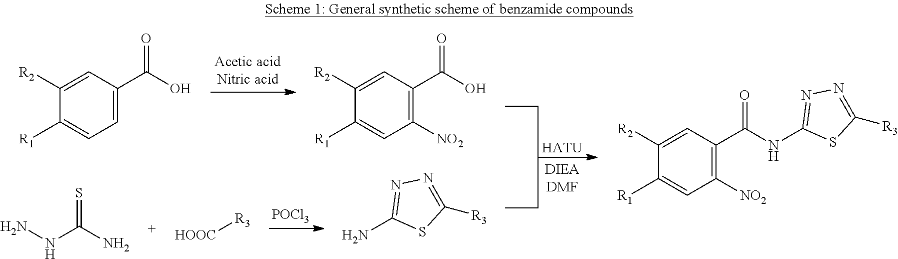

### Synthesis of Benzamide Compounds.

### Representative Procedure for Nitration of Benzoic Acid (Method A).

Nitrobenzoic acid was synthesized by the method described previously^{[3]} with slight modifications.

### 4,5-diethoxy-2-nitrobenzoic acid

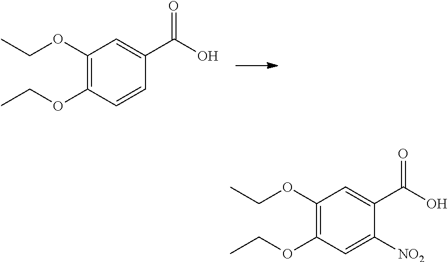 [SMILES: CCOc1ccc(C(=O)O)cc1OCC ; CCOc1cc(C(=O)O)c([N+](=O)[O-])cc1OCC]

A flask immersed in a room-temperature water bath was charged with 3,4-diethoxybenzoic acid (1.29 g. 6.1 mmol) and acetic acid (glacial, 5.2 mL). HNO₃ (70%, 5.4 mL) was slowly added and stirred for 60 min at room temperature. The reaction was quenched upon addition of ice. A yellow precipitate formed that was filtered and washed with H₂O. Recrystallization from DCM gave 4,5-diethoxy-2-nitrobenzoic acid as an off-white solid (424 mg, 1.7 mmol, 27%). ¹H NMR (600 MHz, CDCl₃) δ 10.29 (s, 1H), 7.37 (s, 1H), 7.24 (s, 1H), 4.24-4.17 (m, 4H), 1.51 (t, J=7.0 Hz, 6H). ¹³C NMR (151 MHz, CDCl₃) δ 170.6, 151.6, 150.9, 142.2, 119.0, 112.5, 108.1, 65.4, 65.4, 14.5, 14.4. HRMS (+) calcd for (M+H)⁺ 256.0816. Found 256.0818.

### Representative Procedure for Amide Coupling (Method B).

### 4,5-diethoxy-2-nitro-N-(5-(pentan-3-yl)-1,3,4-thiadiazol-2-yl)benzamide

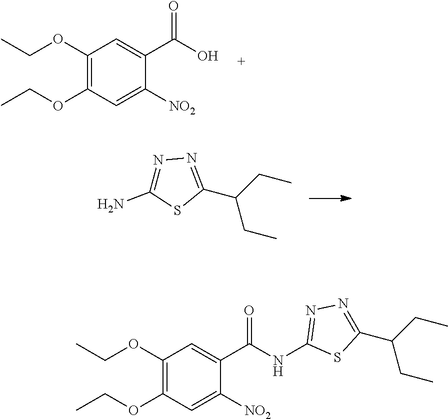 [SMILES: CCOc1cc(C(=O)O)c([N+](=O)[O-])cc1OCC ; CCC(CC)c1nnc(N)s1 ; CCOc1cc(C(=O)Nc2nnc(C(CC)CC)s2)c([N+](=O)[O-])cc1OCC]

To a DMF solution of 4,5-diethoxy-2-nitrobenzoic acid (75 mg, 299 μmol, 1.0 equiv) was added 1-[Bis(dimethylamino)methylene]-1H-1,2,3-triazolo[4,5-b]pyridinium 3-oxid hexafluorophosphate (HATU) (598 μmol, 2.0 equiv), and stirred 10 min. To this solution was added 5-(pentan-3-yl)-1,3,4-thiadiazol-2-amine (104 mg, 598 μmol, 2.0 equiv), followed by diisopropylethylamine (0.5 mL), and stirred at RT overnight. To this solution was added saturated NaHCO₃ aqueous solution, and extracted with diethylether three times. The organic layer was combined, washed with saturated NaHCO₃ aqueous solution and brine, dried over MgSO₄, filtered and concentrated in vacuo. Purification by flash column chromatography (DCM->DCM:MeOH=20:1) followed by recrystallization from DCM/acetone/hexane gave 4,5-diethoxy-2-nitro-N-(5-(pentan-3-yl)-1,3,4-thiadiazol-2-yl)benzamide as an off-white powder (88 mg, 215 μmol, 72%). ¹H NMR (600 MHz, DMSO-d₆) δ 13.03 (s, 1H), 7.69 (s, 1H), 7.37 (s, 1H), 4.24-4.18 (m, 4H), 2.99 (tt, J=8.9, 5.3 Hz, 1H), 1.83-1.75 (m, 2H), 1.73-1.63 (m, 2H), 1.39-1.36 (m, 6H), 0.85 (t, J=7.4 Hz, 6H). ¹³C NMR (151 MHz, DMSO) δ 168.6, 164.4, 158.1, 152.3, 148.8, 139.0, 124.3, 112.2, 108.2, 65.0, 64.8, 43.9, 28.2, 14.39, 14.37, 11.6. HRMS (+) calcd for (M+H)⁺ 409.1540. Found 409.1540. Purity (HPLC-UV): >99% (^{t}R=9.7 min).

### 4,5-dimethoxy-2-nitro-N-(5-(pentan-3-yl)-1,3,4-thiadiazol-2-yl)benzamide (1a)

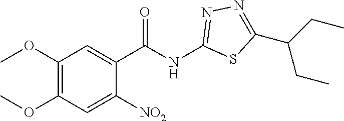 [SMILES: CCC(CC)c1nnc(NC(=O)c2cc(OC)c(OC)cc2[N+](=O)[O-])s1]

The title compound was synthesized by the method B with slight modifications. N-(3-Dimethylaminopropyl)-N′-ethylcarbodiimide hydrochloride (EDCl) was used instead of HATU, and DCM was used as a solvent both for reaction and extraction. Purification by flash column chromatography (DCM->DCM:MeOH=20:1) gave the title compound as a yellowish solid (56 mg, 25%). ¹H NMR (600 MHz, DMSO-d₆) δ 13.04 (s, 1H), 7.72 (s, 1H), 7.39 (s, 1H), 3.93 (s, 3H), 3.92 (s, 3H), 3.00 (tt, J=8.9, 5.3 Hz, 1H), 1.83-1.76 (m, 2H), 1.74-1.63 (m, 2H), 0.85 (t, J=7.4 Hz, 6H). ¹³C NMR (151 MHz, DMSO) δ 168.5, 164.2, 157.9, 152.8, 149.5, 139.0, 124.3, 111.4, 107.2, 56.6, 56.3, 43.7, 28.1, 11.5. LRMS (+) calcd for (M+H)⁺ 381.1 Found 381.1. Purity (HPLC-UV): 95% (tR=8.4 min).

### N-(5-cyclohexyl-1,3,4-thiadiazol-2-yl)-4,5-dimethoxy-2-nitrobenzamide (1c)

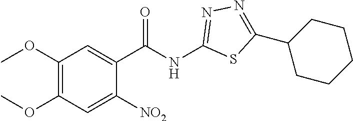 [SMILES: COc1cc(C(=O)Nc2nnc(C3CCCCC3)s2)c([N+](=O)[O-])cc1OC]

Method B (112 mg, off-white powder, 57%). ¹H NMR (600 MHz, DMSO-d₆) δ 13.02 (s, 1H), 7.71 (s, 1H), 7.37 (s, 1H), 3.933 (s, 3H), 3.930 (s, 3H), 3.10 (tt, J=11.3, 3.6 Hz, 1H), 2.10-2.03 (m, 2H), 1.80-1.76 (m, 2H), 1.70-1.67 (m, 1H), 1.58-1.51 (m, 2H), 1.46-1.37 (m, 2H), 1.29-1.26 (m, 1H). ¹³C NMR (151 MHz, DMSO) δ 169.5, 164.3, 157.8, 152.8, 149.5, 139.0, 124.3, 111.4, 107.2, 56.6, 56.3, 38.6, 32.9, 25.3, 25.2. HRMS (+) calcd for (M+H)⁺ 393.1227. Found 393.1228. Purity (HPLC-UV): 99% (^{t}R=8.8 min).

### N-(5-buty-1,3,4-oxadiazol-2-yl)-4,5-dimethoxy-2-nitrobenzamide (1e)

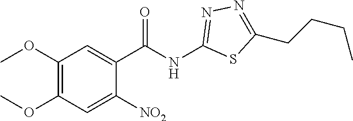 [SMILES: CCCCc1nnc(NC(=O)c2cc(OC)c(OC)cc2[N+](=O)[O-])s1]

Method B (10 mg, brownish powder, 6%). ¹H NMR (600 MHz, DMSO-d₆) δ 7.93 (s, 1H), 7.75 (s, 1H), 7.42 (s, 1H), 3.95 (s, 3H), 3.92 (s, 3H), 2.50 (t, J=7.4 Hz, 2H), 1.50-1.43 (m, 2H), 1.30-1.23 (m, 2H), 0.84 (t, J=7.4 Hz, 3H). ¹³C NMR (151 MHz, DMSO) δ 164.5, 157.8, 153.6, 150.4, 149.7, 138.3, 122.5, 111.2, 106.8, 56.7, 56.3, 26.4, 24.3, 21.0, 13.2. LRMS (+) calcd for (M+H)⁺ 351.1. Found 351.2. Purity (HPLC-UV): 95% (^{t}R=8.1 min).

### 4-ethoxy-5-methoxy-2-nitro-N-(5-(pentan-3-yl)-1,3,4-thiadiazol-2-yl)benzamide (1f)

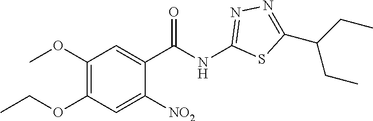 [SMILES: CCOc1cc([N+](=O)[O-])c(C(=O)Nc2nnc(C(CC)CC)s2)cc1OC]

Method B (90 mg, off-white powder, 46%). ¹H NMR (600 MHz, DMSO-d₆) δ 13.04 (s, 1H), 7.69 (s, 1H), 7.39 (s, 1H), 4.20 (q, J=6.9 Hz, 2H), 3.93 (s, 3H), 2.99 (tt, J=9.0, 5.4 Hz, 1H), 1.84-1.74 (m, 2H), 1.73-1.63 (m, 2H), 1.38 (t, J=7.0 Hz, 3H), 0.85 (t, J=7.4 Hz, 6H). ¹³C NMR (151 MHz, DMSO) δ 168.5, 164.3, 157.9, 152.9, 148.7, 139.0, 124.1, 111.5, 107.9, 64.7, 56.6, 43.7, 28.1, 14.3, 11.5. LRMS (+) calcd for (M+H)⁺ 395.1. Found 395.1. Purity (HPLC-UV): >99% (tR=9.5 min).

### 4-butoxy-5-methoxy-2-nitrobenzoic acid

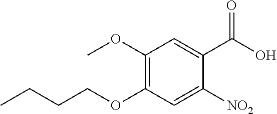 [SMILES: CCCCOc1cc([N+](=O)[O-])c(C(=O)O)cc1OC]

Method A (55 mg, off-white powder, 55%). ¹H NMR (500 MHz, CDCl₃) δ 7.37 (s, 1H), 7.24 (s, 1H), 4.11 (t, J=6.9 Hz, 2H), 3.98 (s, 3H), 1.90-1.86 (m, 2H), 1.56-1.48 (m, 2H), 1.00 (t, J=7.3 Hz, 3H). LRMS (−) calcd for (M−H)⁻ 268.1. Found 268.2.

### 4-butoxy-5-methoxy-2-nitro-N-(5-(pentan-3-yl)-1,3,4-thiadiazol-2-yl)benzamide (1 g)

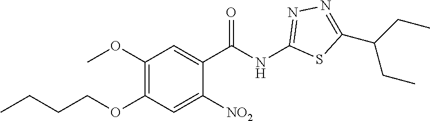 [SMILES: CCCCOc1cc([N+](=O)[O-])c(C(=O)Nc2nnc(C(CC)CC)s2)cc1OC]

Method B (35 mg, off-white powder, 41%). 1H NMR (600 MHz, DMSO-d₆) δ 13.03 (s, 1H), 7.70 (s, 1H), 7.38 (s, 1H), 4.14 (t, J=6.5 Hz, 2H), 3.93 (s, 3H), 2.99 (tt, J=8.9, 5.4 Hz, 1H), 1.84-1.70 (m, 4H), 1.72-1.62 (m, 2H), 1.50-1.40 (m, 2H), 0.95 (t, J=7.4 Hz, 3H), 0.85 (t, J=7.4 Hz, 6H). ¹³C NMR (151 MHz, DMSO) b 168.6, 164.4, 158.0, 153.0, 149.0, 139.2, 124.2, 111.6, 108.1, 68.8, 56.7, 43.9, 30.4, 28.2, 18.7, 13.7, 11.6. LRMS (+) calcd for (M+H)⁺ 423.2. Found 423.1. Purity (HPLC-UV): 97% (tR=10.7 min).

### 4-(hexyloxy)-5-methoxy-2-nitrobenzoic acid

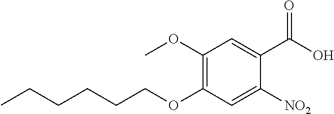 [SMILES: CCCCCCOc1cc([N+](=O)[O-])c(C(=O)O)cc1OC]

Method A (87 mg, off-white powder, 74%). ¹H NMR (500 MHz, CDCl₃) δ 7.37 (s, 1H), 7.21 (s, 1H), 4.10 (t, J=6.5 Hz, 2H), 3.98 (s, 3H), 1.91-1.85 (m, 2H), 1.48-1.44 (m, 2H), 1.37-1.34 (m, 4H), 0.92 (d, J=6.6 Hz, 3H). LRMS (−) calcd for (M−H)⁻ 296.1. Found 296.1.

### 4-(hexyloxy)-5-methoxy-2-nitro-N-(5-(pentan-3-yl)-1,3,4-thiadiazol-2-y)benzamide

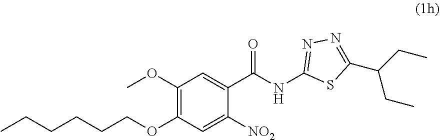 [SMILES: CCCCCCOc1cc([N+](=O)[O-])c(C(=O)Nc2nnc(C(CC)CC)s2)cc1OC]

Method B (22 mg, off-white powder, 24%). ¹H NMR (600 MHz, DMSO-d₆) δ 13.03 (s, 1H), 7.70 (s, 1H), 7.38 (s, 1H), 4.13 (t, J=6.6 Hz, 2H), 3.93 (s, 3H), 2.99 (tt, J=8.9, 5.4 Hz, 1H), 1.84-1.72 (m, 4H), 1.70-1.65 (m, 2H), 1.47-1.39 (m, 2H), 1.37-1.27 (m, 4H), 0.90-0.83 (m, 9H). ¹³C NMR (151 MHz, DMSO) δ 168.5, 164.2, 157.9, 152.9, 148.8, 139.0, 124.1, 111.5, 108.0, 68.9, 56.6, 43.7, 39.8, 30.8, 28.1, 24.9, 21.9, 13.8, 11.5. HRMS (+) calcd for (M+H)⁺ 451.2010. Found 451.2013. Purity (HPLC-UV): 96% (^{t}R=12.2 min).

### 4-isopropoxy-5-methoxy-2-nitrobenzoic acid

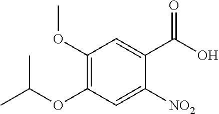 [SMILES: COc1cc(C(=O)O)c([N+](=O)[O-])cc1OC(C)C]

Method A (155 mg, yellowish solid, 53%). ¹H NMR (600 MHz, CDCl₃) δ 7.38 (s, 1H), 7.23 (s, 1H), 4.73-4.61 (m, 1H), 3.97 (s, 3H), 1.44 (d, J=6.1 Hz, 6H). ¹³C NMR (151 MHz, CDCl₃) δ 169.1, 152.8, 149.7, 142.4, 118.9, 111.8, 109.4, 72.4, 56.6, 21.7.

### 4-isopropoxy-5-methoxy-2-nitro-N-(5-(pentan-3-yl)-1,3,4-thiadiazol-2-yl)benzamide

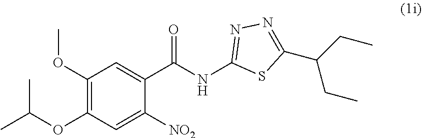 [SMILES: CCC(CC)c1nnc(NC(=O)c2cc(OC)c(OC(C)C)cc2[N+](=O)[O-])s1]

Method B (31 mg, yellowish powder, 18%). ¹H NMR (600 MHz, DMSO-d₆) δ 13.01 (s, 1H), 7.70 (s, 1H), 7.38 (s, 1H), 4.81 (hept, J=6.0 Hz, 1H), 3.92 (s, 3H), 2.99 (tt, J=8.9, 5.4 Hz, 1H), 1.83-1.76 (m, 2H), 1.72-1.64 (m, 2H), 1.32 (d, J=6.0 Hz, 6H), 0.85 (t, J=7.4 Hz, 6H). ¹³C NMR (151 MHz, DMSO) δ 168.5, 164.1, 157.9, 153.6, 147.5, 139.1, 124.0, 111.8, 109.5, 71.3, 56.5, 43.7, 28.1, 21.4, 11.5. HRMS (+) calcd for (M+H)⁺ 409.1540. Found 409.1541. Purity (HPLC-UV): 98% (^{t}R=9.6 min).

### 4-(cyclopentyloxy)-5-methoxy-2-nitrobenzoic acid

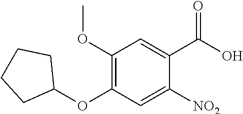 [SMILES: COc1cc(C(=O)O)c([N+](=O)[O-])cc1OC1CCCC1]

Method A (87 mg, yellowish powder, 73%). ¹H NMR (500 MHz, CDCl₃) δ 7.36 (s, 1H), 7.22 (d, J=2.8 Hz, 1H), 4.87-4.84 (m, 1H), 3.96 (s, 3H), 2.04-1.97 (m, 2H), 1.95-1.88 (m, 2H), 1.88-1.85 (m, 2H), 1.71-1.61 (m, 2H). LRMS (−) calcd for (M−H)⁻ 280.1. Found 280.0.

### 4-(cyclopentyloxy)-5-methoxy-2-nitro-N-(5-(pentan-3-yl)-1,3,4-thiadiazol-2-yl)benzamide (1j)

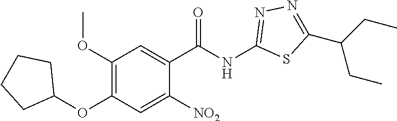 [SMILES: CCC(CC)c1nnc(NC(=O)c2cc(OC)c(OC3CCCC3)cc2[N+](=O)[O-])s1]

Method B (19 mg, off-white powder, 22%). ¹H NMR (600 MHz, DMSO-d₆) δ 13.01 (s, 1H), 7.66 (s, 1H), 7.37 (s, 1H), 5.03-5.00 (m, 1H), 3.91 (s, 3H), 2.99 (tt, J=9.3, 5.3 Hz, 1H), 2.00-1.95 (m, 2H), 1.83-1.66 (m, 8H), 1.66-1.58 (m, 2H), 0.85 (t, J=7.4 Hz, 6H). ¹³C NMR (151 MHz, DMSO) δ 168.5, 164.2, 158.0, 153.4, 147.7, 139.0, 123.9, 111.6, 109.1, 80.5, 56.6, 43.7, 31.9, 28.1, 23.5, 11.5. HRMS (+) calcd for (M+H)⁺ 435.1697. Found 435.1697. Purity (HPLC-UV): 98% (^{t}R=10.7 min).

### N-(4-ethylphenyl)-4,5-dimethoxy-2-nitrobenzamide

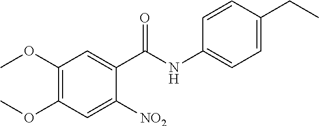 [SMILES: CCc1ccc(NC(=O)c2cc(OC)c(OC)cc2[N+](=O)[O-])cc1]

Method B (233 mg, off-white crystals, 71%). ¹H NMR (600 MHz, DMSO-d₆) δ 10.41 (s, 1H), 7.69 (s, 1H), 7.58-7.55 (m, 2H), 7.25 (s, 1H), 7.20-7.17 (m, 2H), 3.94 (s, 3H), 3.92 (s, 3H), 2.58 (q, J=7.6 Hz, 2H), 1.17 (t, J=7.6 Hz, 3H). ¹³C NMR (151 MHz, DMSO) δ 163.8, 152.9, 148.8, 139.1, 138.6, 136.6, 127.8, 127.3, 119.5, 110.9, 107.1, 56.5, 56.2, 27.6, 15.7. LRMS (+) calcd for (M+H)⁺ 331.1. Found 331.2. Purity (HPLC-UV): >99% (tR=8.6 min).

### Ethyl 2-(5-(4,5-dimethoxy-2-nitrobenzamido)-1,3,4-thiadiazol-2-yl)acetate

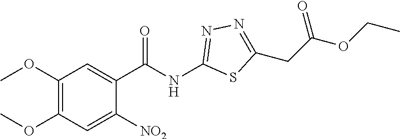 [SMILES: CCOC(=O)Cc1nnc(NC(=O)c2cc(OC)c(OC)cc2[N+](=O)[O-])s1]

Method B (181 mg, yellowish crystals, 46%). ¹H NMR (600 MHz, DMSO-d₆) δ 13.10 (s, 1H), 7.73 (s, 1H), 7.40 (s, 1H), 4.26 (s, 2H), 4.18 (q, J=7.1 Hz, 2H), 3.94 (s, 6H), 1.24 (t, J=7.1 Hz, 3H). ¹³C NMR (151 MHz, DMSO) δ 168.9, 164.5, 159.7, 157.1, 153.0, 149.7, 139.1, 124.3, 111.5, 107.4, 61.2, 56.8, 56.4, 35.0, 14.1. LRMS (+) calcd for (M+H)⁺ 397.1. Found 397.0. Purity (HPLC-UV): 98% (^{t}R=6.7 min).

### 5-(heptan-4-yl)-1,3,4-thiadiazol-2-amine

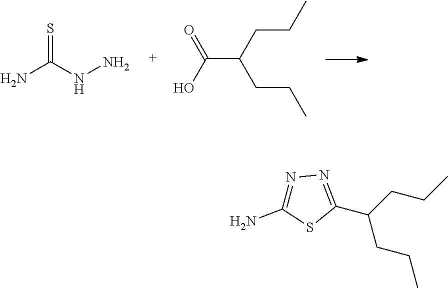

The title compound was synthesized by the method described previously with slight modifications^{[4]}. To an ice-cooled mixture of thiosemicarbazide (0.450 g, 4.95 mmol) and 2-propylpentanoic acid (0.713 g, 4.95 mmol), an excess of phosphorus oxychloride (0.9 mL, 9.9 mmol) was added slowly. Subsequently, the temperature was raised gradually to 75° C. The reaction was kept at this temperature and stirred for 1 h. After cooling to RT, ice-water was added, and the mixture was stirred for an additional hour. The solution was extracted with ethyl acetate. The organic layer was combined, washed with aqueous saturated NaHCO₃ solution and brine, dried over MgSO₄, filtered, and concentrated in vacuo to give the title compound as an off-white solid (340 mg, 35%). ¹H NMR (500 MHz, DMSO-d₆) δ 9.71 (s, 2H), 3.00 (tt, J=9.3, 5.3 Hz, 1H), 1.65-1.50 (m, 4H), 1.28-1.18 (m, 4H), 0.86 (t, J=7.2 Hz, 6H). ¹³C NMR (151 MHz, DMSO) δ 168.8, 162.8, 40.2, 36.5, 19.5, 13.6. LRMS (+) calcd for (M+H)⁺ 200.1. Found 200.1.

### N-(5-(heptan-4-yl)-1,3,4-thiadiazol-2-yl)-4,5-dimethoxy-2-nitrobenzamide

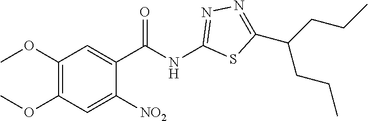 [SMILES: CCCC(CCC)c1nnc(NC(=O)c2cc(OC)c(OC)cc2[N+](=O)[O-])s1]

Method B (95 mg, off-white powder, 47%). ¹H NMR (600 MHz, DMSO-d₆) δ 13.05 (s, 1H), 7.72 (s, 1H), 7.39 (s, 1H), 3.94 (s, 3H), 3.93 (s, 3H), 3.17 (tt, J=9.3, 5.3 Hz, 1H), 1.75-1.61 (m, 4H), 1.29-1.18 (m, 4H), 0.88 (t, J=7.3 Hz, 6H). ¹³C NMR (151 MHz, DMSO) δ 168.9, 164.2, 157.9, 152.8, 149.5, 139.0, 124.3, 111.4, 107.2, 56.6, 56.3, 37.8, 30.9, 19.8, 13.6. LRMS (+) calcd for (M+H)⁺ 409.2. Found 409.1. Purity (HPLC-UV): 99% (^{t}R=10.6 min).

### 5-ethoxy-4-methoxy-2-nitro-N-(5-(pentan-3-yl)-1,3,4-thiadiazol-2-yl)benzamide

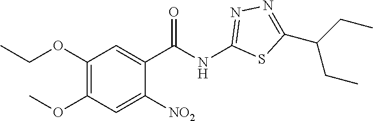 [SMILES: CCOc1cc(C(=O)Nc2nnc(C(CC)CC)s2)c([N+](=O)[O-])cc1OC]

Method B (46 mg, off-white powder, 23%). ¹H NMR (600 MHz, DMSO-d₆) δ 13.03 (s, 1H), 7.71 (s, 1H), 7.37 (s, 1H), 4.21 (q, J=7.0 Hz, 2H), 3.93 (s, 3H), 2.99 (tt, J=8.9, 5.3 Hz, 1H), 1.81-1.77 (m, 2H), 1.70-1.67 (m, 2H), 1.37 (t, J=7.0 Hz, 3H), 0.85 (t, J=7.4 Hz, 6H). ¹³C NMR (151 MHz, DMSO) δ 168.5, 164.2, 157.9, 152.1, 149.5, 138.8, 124.3, 111.9, 107.3, 64.9, 56.2, 43.7, 28.1, 14.3, 11.5. LRMS (+) calcd for (M+H)⁺ 395.1. Found 395.1. Purity (HPLC-UV): >99% (^{t}R=9.6 min).

### 2-nitro-4,5-dipropoxybenzoic acid

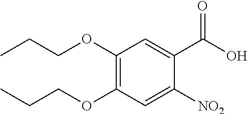 [SMILES: CCCOc1cc(C(=O)O)c([N+](=O)[O-])cc1OCCC]

Method A (424 mg, yellowish solid, 27%). ¹H NMR (400 MHz, CDCl₃) δ 7.37 (s, 1H), 7.22 (s, 1H), 4.10-4.04 (m, 4H), 1.96-1.85 (m, 4H), 1.10-1.04 (m, 6H).

### 2-nitro-N-(5-(pentan-3-yl)-1,3,4-thiadiazol-2-yl)-4,5-dipropoxybenzamide

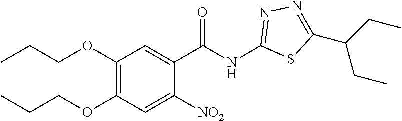 [SMILES: CCCOc1cc(C(=O)Nc2nnc(C(CC)CC)s2)c([N+](=O)[O-])cc1OCCC]

Method B (33 mg, off-white powder, 33%). ¹H NMR (600 MHz, DMSO-d₆) δ 13.01 (s, 1H), 7.69 (s, 1H), 7.38 (s, 1H), 4.13-4.10 (m, 4H), 3.03-2.95 (m, 1H), 1.83-1.73 (m, 6H), 1.72-1.64 (m, 2H), 1.01-0.98 (m, 6H), 0.85 (t, J=7.4 Hz, 6H). ¹³C NMR (151 MHz, DMSO) δ 168.5, 164.2, 157.9, 152.5, 148.9, 138.8, 124.2, 112.3, 108.4, 70.5, 70.3, 43.7, 28.1, 21.7, 21.6, 11.5, 10.1, 10.1. LRMS (+) calcd for (M+H)⁺ 437.2. Found 436.9. Purity (HPLC-UV): 98% (^{t}R=11.5 min).

### 4-methoxy-2-nitro-N-(5-(pentan-3-yl)-1,3,4-thiadiazol-2-yl)benzamide

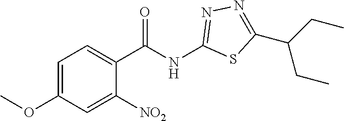 [SMILES: CCC(CC)c1nnc(NC(=O)c2ccc(OC)cc2[N+](=O)[O-])s1]

Method B (49 mg, white powder, 35%). ¹H NMR (600 MHz, DMSO-d₆) δ 13.15 (s, 1H), 7.82 (d, J=8.6 Hz, 1H), 7.65 (d, J=2.6 Hz, 1H), 7.42 (dd, J=8.6, 2.6 Hz, 1H), 3.93 (s, 3H), 2.98 (ddd, J=10.6, 9.1, 5.3 Hz, 1H), 1.78 (dtd, J=14.7, 7.4, 5.4 Hz, 2H), 1.73-1.62 (m, 2H), 0.85 (t, J=7.4 Hz, 6H). ¹³C NMR (151 MHz, DMSO) δ 168.6, 164.0, 161.3, 158.1, 148.7, 131.3, 121.5, 118.8, 109.8, 56.4, 43.9, 28.1, 11.6.

LRMS (+) calcd for (M+H)⁺ 351.1. Found 351.1. Purity (HPLC-UV): 99% (^{t}R=8.6 min).

### 5-methoxy-2-nitro-N-(5-(pentan-3-yl)-1,3,4-thiadiazol-2-yl)benzamide

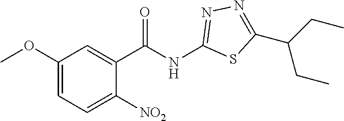 [SMILES: CCC(CC)c1nnc(NC(=O)c2cc(OC)ccc2[N+](=O)[O-])s1]

Method B (56 mg, white powder, 40%). ¹H NMR (600 MHz, DMSO-d₆) δ 13.08 (s, 1H), 8.22 (d, J=9.2 Hz, 1H), 7.36 (d, J=2.8 Hz, 1H), 7.27 (dd, J=9.2, 2.8 Hz, 1H), 3.93 (s, 3H), 3.01-2.98 (m, 1H), 1.82-1.79 (m, 2H), 1.74-1.63 (m, 2H), 0.85 (t, J=7.4 Hz, 6H). ¹³C NMR (151 MHz, DMSO) δ 168.7, 164.3, 163.6, 157.8, 138.6, 133.2, 127.1, 116.2, 114.7, 56.7, 43.8, 28.2, 11.6. LRMS (+) calcd for (M+H)⁺ 351.1.

Found 351.1. Purity (HPLC-UV): 99% (^{t}R=8.5 min).

### 3,4,5-trimethoxy-2-nitro-N-(5-(pentan-3-yl)-1,3,4-thiadiazol-2-yl)benzamide

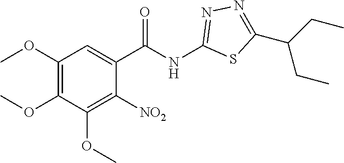 [SMILES: CCC(CC)c1nnc(NC(=O)c2cc(OC)c(OC)c(OC)c2[N+](=O)[O-])s1]

Method B (127 mg, off-white powder, 62%). ¹H NMR (600 MHz, DMSO-d₆) δ 13.39 (broad s, 1H), 7.54 (s, 1H), 3.98 (s, 3H), 3.91 (s, 6H), 2.97 (tt, J=8.9, 5.4 Hz, 1H), 1.77 (dtd, J=14.8, 7.4, 5.4 Hz, 2H), 1.72-1.61 (m, 2H), 0.83 (t, J=7.4 Hz, 6H). ¹³C NMR (151 MHz, DMSO) δ 168.4, 162.2, 158.6, 154.1, 145.5, 144.5, 138.2, 121.5, 108.4, 62.5, 60.9, 56.7, 43.8, 28.0, 11.4. LRMS (+) calcd for (M+H)⁺ 411.1. Found 411.1. Purity (HPLC-UV): 98% (^{t}R=10.1 min).

### 5-methoxy-2-nitro-4-propoxybenzoic acid

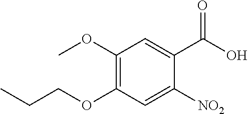 [SMILES: CCCOc1cc([N+](=O)[O-])c(C(=O)O)cc1OC]

Method A (52 mg, off-white powder, 43%). ¹H NMR (500 MHz, CDCl₃) δ 7.37 (s, 1H), 7.23 (s, 1H), 4.07 (t, J=6.8 Hz, 2H), 3.98 (s, 3H), 1.92 (m, 2H), 1.07 (t, J=7.3 Hz, 3H). LRMS (−) calcd for (M−H)⁻ 254.1. Found 254.0.

### 5-methoxy-2-nitro-N-(5-(pentan-3-yl)-1,3,4-thiadiazol-2-yl)-4-propoxybenzamide

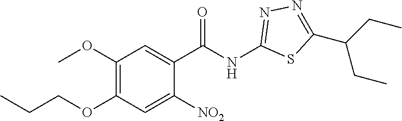 [SMILES: CCCOc1cc([N+](=O)[O-])c(C(=O)Nc2nnc(C(CC)CC)s2)cc1OC]

Method B (25 mg, off-white powder, 31%). ¹H NMR (600 MHz, DMSO-d₆) δ 13.03 (s, 1H), 7.69 (s, 1H), 7.39 (s, 1H), 4.10 (t, J=6.6 Hz, 2H), 3.93 (s, 3H), 2.99 (tt, J=8.9, 5.4 Hz, 1H), 1.84-1.74 (m, 4H), 1.71-1.64 (m, 2H), 1.00 (t, J=7.4 Hz, 3H), 0.85 (t, J=7.4 Hz, 6H). ¹³C NMR (151 MHz, DMSO) δ 168.5, 164.2, 157.9, 152.9, 148.8, 139.0, 124.1, 111.5, 108.0, 70.4, 56.6, 43.7, 28.1, 21.7, 11.5, 10.2. LRMS (+) calcd for (M+H)⁺ 409.2. Found 409.0. Purity (HPLC-UV): 98% (^{t}R=10.5 min).

### 4-isobutoxy-5-methoxy-2-nitrobenzoic acid

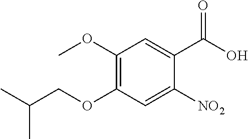 [SMILES: COc1cc(C(=O)O)c([N+](=O)[O-])cc1OCC(C)C]

Method A (56 mg, off-white powder, 47%). ¹H NMR (500 MHz, CDCl₃) δ 7.36 (d, J=2.8 Hz, 1H), 7.23 (d, J=2.7 Hz, 1H), 3.98 (s 3H), 3.85 (d, J=6.7 Hz, 2H), 2.24-2.15 (m, 1H), 1.06 (d, J=6.7 Hz, 6H). LRMS (−) calcd for (M−H)⁻ 268.1. Found 268.1.

### 4-isobutoxy-5-methoxy-2-nitro-N-(5-(pentan-3-yl)-1,3,4-thiadiazol-2-yl)benzamide

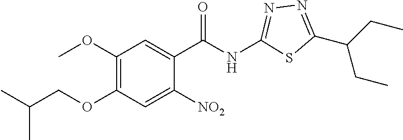 [SMILES: CCC(CC)c1nnc(NC(=O)c2cc(OC)c(OCC(C)C)cc2[N+](=O)[O-])s1]

Method B (37 mg, yellowish crystals, 42%). ¹H NMR (600 MHz, DMSO-d₆) δ 13.03 (s, 1H), 7.70 (s, 1H), 7.39 (s, 1H), 3.96-3.90 (m, 5H), 2.99 (tt, J=8.9, 5.4 Hz, 1H), 2.11-2.04 (m, 1H), 1.83-1.75 (m, 2H), 1.72-1.64 (m, 2H), 1.00 (d, J=6.7 Hz, 6H), 0.85 (t, J=7.4 Hz, 6H). ¹³C NMR (151 MHz, DMSO) δ 168.6, 164.3, 158.0, 153.1, 149.0, 139.1, 124.2, 111.6, 108.1, 75.1, 56.8, 43.8, 28.2, 27.6, 18.9, 11.6.

LRMS (+) calcd for (M+H)⁺ 423.2. Found 423.1. Purity (HPLC-UV): 98% (^{t}R=10.8 min).

### 4-(isopentyloxy)-5-methoxy-2-nitrobenzoic acid

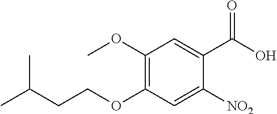 [SMILES: COc1cc(C(=O)O)c([N+](=O)[O-])cc1OCCC(C)C]

Method A (97 mg, off-white solid, 33%). ¹H NMR (500 MHz, CDCl₃) δ 7.38 (s, 1H), 7.23 (s, 1H), 4.13 (t, J=6.7 Hz, 2H), 3.98 (s, 3H), 1.88-1.79 (m, 1H), 1.79-1.76 (m, 2H), 0.99 (d, J=6.5 Hz, 6H). LRMS (−) calcd for (M−H)⁻ 282.1. Found 282.2.

### 4-(isopentyloxy)-5-methoxy-2-nitro-N-(5-(pentan-3-yl)-1,3,4-thiadiazol-2-yl)benzamide

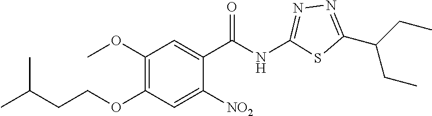 [SMILES: CCC(CC)c1nnc(NC(=O)c2cc(OC)c(OCCC(C)C)cc2[N+](=O)[O-])s1]

Method B (20 mg, yellowish powder, 23%). ¹H NMR (600 MHz, DMSO-d₆) δ 13.03 (s, 1H), 7.73 (s, 1H), 7.38 (s, 1H), 4.17 (t, J=6.7 Hz, 2H), 3.93 (s, 3H), 2.99 (tt, J=8.8, 5.4 Hz, 1H), 1.83-1.75 (m, 3H), 1.72-1.64 (m, 4H), 0.95 (d, J=6.6 Hz, 6H), 0.85 (t, J=7.4 Hz, 6H). ¹³C NMR (151 MHz, DMSO) δ 168.5, 164.2, 157.9, 152.9, 148.8, 139.1, 124.1, 111.5, 108.0, 67.4, 56.6, 43.7, 37.0, 28.1, 24.4, 22.3, 11.5.

LRMS (+) calcd for (M+H)⁺ 437.2. Found 436.8. Purity (HPLC-UV): 96% (^{t}R=11.3 min).

### methyl 4-(sec-butoxy)-3-methoxybenzoate

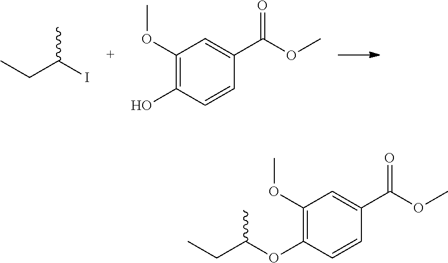

The title compound was synthesized by the method described previously with slight modifications^{[5]}. To a DMF solution of methyl 4-hydroxy-3-methoxybenzoate (440 mg, 2.4 mmol, 1 equiv) was slowly added sodium hydride (60% dispersion in mineral oil, 150 mg, 3.75 mmol, 1.56 equiv) followed by 2-iodobutane (1.4 mL, 12 mmol, 5 equiv). The solution was heated to 65° C. and stirred 4 h. After cooling to rt, aqueous saturated LiCl was added and extracted with ethyl acetate. The organic layer was combined and washed with brine, dried over MgSO₄, filtered, and concentrated in vacuo. Purification by flash column chromatography (Hexane: ethyl acetate=5:1) gave the title compound as a white powder (200 mg, 35%). ¹H NMR (500 MHz, CDCl₃) δ 7.64 (dd, J=8.5, 2.0 Hz, 1H), 7.55 (d, J=2.0 Hz, 1H), 6.88 (d, J=8.5 Hz, 1H), 4.42-4.35 (m, 1H), 3.90-3.88 (m, 6H), 1.89-1.77 (m, 1H), 1.74-1.60 (m, 1H), 1.35 (d, J=6.1 Hz, 3H), 0.99 (t, J=7.5 Hz, 3H). ¹³C NMR (126 MHz, DMSO) δ 166.9, 151.9, 149.7, 123.4, 122.4, 113.4, 112.8, 76.5, 56.1, 51.9, 29.1, 19.2, 9.8.

### 4-(sec-butoxy)-3-methoxybenzoic acid

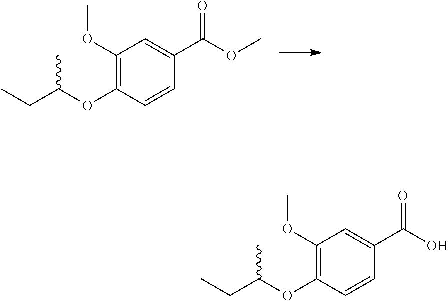

To a solution of methyl 4-(sec-butoxy)-3-methoxybenzoate (200 mg, 0.84 mmol) in methanol (20 mL) was added 2M NaOHaq solution (10 mL) and stirred for 1 hour. To the reaction mixture was added 1 M HCl aqueous solution (50 mL) and extracted with ethyl acetate three times. The organic layer was combined and washed with brine, dried over MgSO₄, filtered, and concentrated in vacuo to give the title compound as an off-white solid (180 mg, 96%). ¹H NMR (500 MHz, CDCl₃) δ 12.13 (s, 1H), 7.75 (dd, J=8.2, 2.0 Hz, 1H), 7.62 (d, J=2.0 Hz, 1H), 6.91 (d, J=8.4 Hz, 1H), 4.44-4.38 (m, 1H), 3.92 (s, 3H), 1.90-1.81 (m, 1H), 1.72-1.63 (m, 1H), 1.37 (d, J=6.1 Hz, 3H), 1.00 (t, J=7.5 Hz, 3H).

### 4-(sec-butoxy)-5-methoxy-2-nitrobenzoic acid

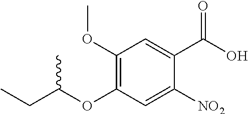 [SMILES: CCC(C)Oc1cc([N+](=O)[O-])c(C(=O)O)cc1OC]

Method A (51 mg, off-white solid, 24%). ¹H NMR (600 MHz, CDCl₃) δ 7.75 (s, 1H), 7.36 (s, 1H), 7.26 (s, 1H), 4.45-4.40 (m, 1H), 3.98 (s, 3H), 1.90-1.82 (m, 1H), 1.74-1.70 (m, 1H), 1.39 (d, J=6.1 Hz, 3H), 1.01 (t, J=7.4 Hz, 3H). ¹³C NMR (151 MHz, CDCl₃) δ 170.2, 152.8, 150.2, 142.5, 118.6, 111.9, 109.4, 77.6, 56.6, 28.9, 19.0, 9.7.

### 4-(sec-butoxy)-5-methoxy-2-nitro-N-(5-(pentan-3-yl)-1,3,4-thiadiazol-2-yl)benzamide

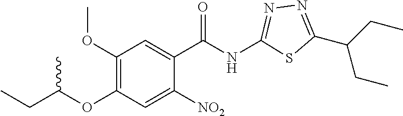 [SMILES: CCC(C)Oc1cc([N+](=O)[O-])c(C(=O)Nc2nnc(C(CC)CC)s2)cc1OC]

Method B (30 mg, yellowish powder, 37%). ¹H NMR (600 MHz, DMSO-d₆) b 13.01 (s, 1H), 7.70 (s, 1H), 7.38 (s, 1H), 4.64-4.59 (m, 1H), 3.93 (s, 3H), 2.99 (tt, J=8.9, 5.3 Hz, 1H), 1.84-1.58 (m, 6H), 1.28 (d, J=6.1 Hz, 3H), 0.94 (t, J=7.4 Hz, 3H), 0.85 (t, J=7.4 Hz, 6H). ¹³C NMR (151 MHz, DMSO) b 168.5, 164.2, 157.9, 153.6, 147.8, 139.1, 123.9, 111.8, 109.5, 76.1, 56.6, 43.7, 28.2, 28.1, 18.6, 11.5, 9.3.

HRMS (+) calcd for (M+H)⁺ 423.1697. Found 423.1698. Purity (HPLC-UV): >99% (^{t}R=10.4 min).

### methyl 3-methoxy-4-(pentan-3-yloxy)benzoate

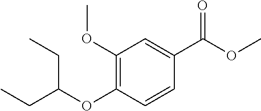 [SMILES: CCC(CC)Oc1ccc(C(=O)OC)cc1OC]

The title compound was synthesized by the method described for methyl 4-(sec-butoxy)-3-methoxybenzoate using 3-bromopentane. It should be noted that a catalytic amount of Nal was added to the reaction mixture. (334 mg, off-white powder, 37%). ¹H NMR (500 MHz, CDCl₃) δ 7.64 (d, J=8.2 Hz, 1H), 7.56 (d, J=1.9 Hz, 1H), 6.88 (d, J=8.4 Hz, 1H), 4.22-4.18 (m, 1H), 3.90 (s, 3H), 3.88 (s, 3H), 1.79-1.66 (m, 4H), 0.97 (t, J=7.5 Hz, 6H). ¹³C NMR (126 MHz, DMSO) δ 166.7, 152.4, 149.7, 123.3, 122.2, 113.5, 112.8, 81.6, 55.9, 51.7, 26.1, 9.5.

### 3-methoxy-4-(pentan-3-yloxy)benzoic acid

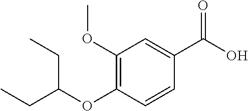 [SMILES: CCC(CC)Oc1ccc(C(=O)O)cc1OC]

The title compound was synthesized by the method described for 4-(sec-butoxy)-3-methoxybenzoic acid from methyl 3-methoxy-4-(pentan-3-yloxy)benzoate. (170 mg, off-white solid, 54%).

¹H NMR (600 MHz, CDCl₃) δ 11.81 (s, 1H), 7.74 (dd, J=8.4, 2.0 Hz, 1H), 7.62 (d, J=2.0 Hz, 1H), 6.91 (d, J=8.6 Hz, 1H), 4.26-4.21 (m, 1H), 3.91 (s, 3H), 1.80-1.65 (m, 4H), 0.98 (t, J=7.4 Hz, 6H). ¹³C NMR (151 MHz, CDCl₃) δ 172.2, 153.4, 149.7, 124.5, 121.4, 113.4, 113.3, 81.9, 56.1, 26.3, 9.7.

### 5-methoxy-2-nitro-4-(pentan-3-yloxy)benzoic acid

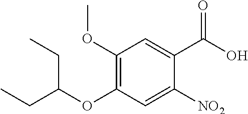 [SMILES: CCC(CC)Oc1cc([N+](=O)[O-])c(C(=O)O)cc1OC]

Method A (29 mg, off-white powder, 14%). ¹H NMR (600 MHz, CDCl₃) δ 7.35 (s, 1H), 7.26 (s, 1H), 4.25 (p, J=5.9 Hz, 1H), 3.98 (s, 3H), 1.76 (qdd, J=7.3, 5.9, 3.9 Hz, 4H), 0.98 (t, J=7.4 Hz, 6H). ¹³C NMR (151 MHz, CDCl₃) δ 170.3, 152.9, 150.7, 142.6, 118.5, 112.0, 109.6, 82.9, 56.6, 26.0, 9.6.

### 5-methoxy-2-nitro-N-(5-(pentan-3-yl)-1,3,4-thiadiazol-2-yl)-4-(pentan-3-yloxy)benzamide

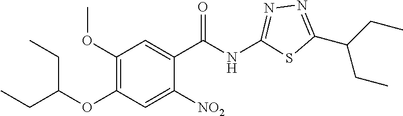 [SMILES: CCC(CC)Oc1cc([N+](=O)[O-])c(C(=O)Nc2nnc(C(CC)CC)s2)cc1OC]

Method B (20 mg, off-white powder, 45%). ¹H NMR (600 MHz, DMSO-d₆) δ 13.00 (s, 1H), 7.71 (s, 1H), 7.39 (s, 1H), 4.48 (p, J=5.8 Hz, 1H), 3.93 (s, 3H), 2.99 (tt, J=8.9, 5.3 Hz, 1H), 1.79 (dtd, J=14.8, 7.3, 5.3 Hz, 2H), 1.73-1.61 (m, 6H), 0.91 (t, J=7.4 Hz, 6H), 0.85 (t, J=7.4 Hz, 6H). ¹³C NMR (151 MHz, DMSO) δ 168.5, 164.2, 157.9, 153.6, 148.3, 139.1, 123.8, 111.9, 109.5, 80.9, 56.6, 43.7, 28.1, 25.2, 11.5, 9.1.

HRMS (+) calcd for (M+H)⁺ 437.1853. Found 437.1855. Purity (HPLC-UV): >99% (^{t}R=11.1 min).

### DOCUMENTS CITED IN EXAMPLES SECTION

[1] M. European Committee for Antimicrobial Susceptibility Testing of the European Society of Clinical, D. Infectious, Clin. Microbiol. Infect. 2003, 9, ix-xv; b) I. Wiegand, K. Hilpert, R. E. W. Hancock, Nat. Protocols 2008, 3, 163-175.
[2] Y. Xiao, K. Gerth, R. Müller, D. Wall, Antimicrob. Agents Chemother. 2012, 56, 2014-2021.
[3] H. F. VanBrocklin, J. K. Lim, S. L. Coffing, D. L. Hom, K. Negash, M. Y. Ono, J. L. Gilmore, I. Bryant, D. J. Riese, J. Med. Chem. 2005, 48, 7445-7456.
[4] S. Ferrari, F. Morandi, D. Motiejunas, E. Nerini, S. Henrich, R. Luciani, A. Venturelli, S. Lazzari, S. Caló, S. Gupta, V. Hannaert, P. A. M. Michels, R. C. Wade, M. P. Costi, J. Med. Chem. 2011, 54, 211-221.
[5] G. Wang, L. Beigelman, A. Truong, C. Napolitano, D. Andreotti, H. He, K. Stein, Ann; PCT/US2014/051642 2015.

While the invention has been described and exemplified in sufficient detail for those skilled in this art to make and use it, various alternatives, modifications, and improvements will be apparent to those skilled in the art without departing from the spirit and scope of the claims.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A compound of formula (1) 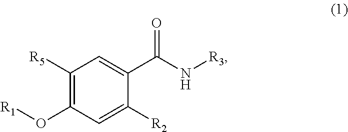
wherein
R₁ is (C1-C6) alkyl, or is a 5-, 6- or 7-membered cycloalkyl;
R₂ is H, NO₂, halo, or trifluoromethyl;
R₃ is a 1,3,4-thiadiazole of formula 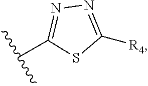
wherein R₄ is (C4-C6) straight or branched chain alkyl, or is a 5-, 6- or 7-membered cycloalkyl;
R₅ is H or (C1-C4)alkoxyl;
or a pharmaceutically acceptable salt thereof.

2. The compound of claim 1, wherein R₁ is isopropyl or cyclopentyl.

3. The compound of claim 1, wherein R₂ is NO₂.

4. The compound of claim 1, wherein R₃ is any one of

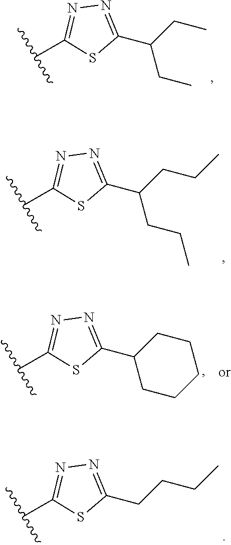

5. A method of inhibiting a bacterial lipoprotein signal peptidase (Lsp), comprising contacting the peptidase with an effective amount or concentration of a compound of claim 1.

6. A method of treatment of a bacterial infection in a patient, comprising administering to the patient an effective dose of a compound of claim 1.

7. A method of screening compounds for inhibitory bioactivity of a bacterial lipoprotein signal peptidase (Lsp), comprising contacting a Lsp peptide FRET substrate, comprising a hexapeptide VTGCAK, with a N-terminal dabsyl quencher and C-terminal EDANS fluorophore, wherein the cysteine residue of the hexapeptide is S-alkylated with a diacylglycerol residue, and a candidate inhibitor compound, then measuring fluorescence from the fluorophore signalling cleavage of the Lsp FRET substrate and its inhibition by the candidate inhibitor.

8. The method of claim 7 wherein screening of multiple compounds are carried out in parallel in a High Throughput Screening format.
